(19) United States
(12) Patent Application Publication
(10) Pub. No.: US 2022/0277814 A1
(43) Pub. Date: Sep. 1, 2022

(54) NUCLEIC ACID CONSTRUCTS AND RELATED METHODS FOR NANOPORE READOUT AND SCALABLE DNA CIRCUIT REPORTING

(71) Applicant: University of Washington, Seattle, WA (US)

(72) Inventor: Jeffrey Matthew Nivala, Seattle, WA (US)

(73) Assignee: University of Washington, Seattle, WA (US)

(21) Appl. No.: 17/630,135

(22) PCT Filed: Jul. 24, 2020
(86) PCT No.: PCT/US20/43382

§ 371 Date: Jan. 25, 2022

Related U.S. Application Data
(60) Provisional application No. 62/879,204, filed on Jul. 26, 2019.

(51) Int. Cl. G16B 50/30; C12Q 1/6825; G16B 30/00; G01N 33/487; G01N 27/447

(52) U.S. Cl. CPC G16B 50/30; C12Q 1/6825; G16B 30/00; G01N 33/48721; G01N 27/44791; B82Y 15/00

(57) ABSTRACT

The disclosure provides compositions, systems, and related method for using nanopore-based detection of nucleic acid displacement circuits. In some embodiments, output strands contain orthogonal barcode sequences that are captured by nanopore systems to produce a unique and recognizable current signal. Machine learning models can be used to differentiate a plurality of current signals and, therefore, monitor detection and quantification of multiple nucleic acid displacement circuits in a single pot reaction.

### CROSS-REFERENCE TO RELATED APPLICATION

This application claims the benefit of U.S. Provisional Application No. 62/879,204, filed Jul. 26, 2019, the disclosure of which is incorporated herein by reference in its entirety.

### STATEMENT OF GOVERNMENT LICENSE RIGHTS

This invention was made with government support under Grant No. 1841188, awarded by the National Science Foundation. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 72243_Sequence_final_2020-07-22.txt. The text file is 9 KB; was created on Jul. 22, 2020; and is being submitted via EFS-Web with the filing of the specification.

### BACKGROUND

As information processing machines approach the nanoscale level, DNA has emerged as a powerful tool for storing and processing information. The predictability of Watson Crick base pairing has enabled the construction of a wide range of DNA computers including amplifiers, Boolean logic gates, chemical reaction networks, oscillators, and neural networks. These circuits rely on a basic molecular mechanism called toehold-mediated DNA strand displacement (DSD). DSD is a competitive hybridization reaction whereby a single stranded DNA (input) displaces an incumbent (output) from a complementary binding partner. Multiple DSD reactions can cascade to create a complex reaction network. Due to the simplicity of this mechanism and its applicability in cell-free settings, DSD circuits have been rapidly scaled up and constitute the largest molecular circuits fully rationally designed by humanity so far.

Readout of DNA circuits typically relies on fluorescence reporters. A DNA strand is labeled with a fluorophore that absorbs light within its absorption band and emits light within its emission band that is detected by optical sensors. Spectrofluorometers or plate readers with fluorescence detection offer great sensitivity and the capability of reporting signals in real-time. However, due to the spectral overlap of fluorophores, the number of unique signals that can be detected in a one-pot reaction are limited. As the complexity of DNA circuits rapidly increases, more scalable detection methods become critical.

Accordingly, despite the advances in DNA circuit technologies, a need remains for DNA circuits that incorporate scalable detection signals that can be detected and differentiated in parallel, e.g., in a single-pot reaction. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of detecting a nucleic acid strand displacement circuit in a nanopore system. The nanopore system comprises a nanopore disposed between a first conductive liquid medium and a second conductive liquid medium, wherein the nanopore comprises a tunnel that provides liquid communication between the first conductive liquid medium and the second conductive liquid medium. The method comprises contacting a double stranded complex with an input strand, wherein the double stranded complex comprises a partner strand hybridized along a first portion of the partner strand to an output strand along a portion of the output strand. Additionally, the method comprises permitting hybridization of the input strand to the partner strand along a second portion of the partner strand that partially overlaps with the first portion of the partner strand, thereby displacing the output strand from the double stranded complex. Additionally, the method comprises translocating the displaced output strand through the nanopore from the first conductive liquid medium towards the second conductive liquid medium. Additionally, the method comprises measuring an ion current through the nanopore when the displaced output strand is in the tunnel to provide a current pattern corresponding to a portion of the displaced output strand. Additionally, the method comprises associating the current pattern with the input strand that displaced the output strand, thereby detecting the nucleic acid strand displacement circuit. In some embodiments, the output strand comprises a barcode sequence, which can be randomly generated or rationally designed to produce a distinct current pattern in the nanopore system. In some embodiments, the translocating, measuring, and associating steps are repeated to quantify the capture events over time for a given output strand species. The method can be scaled up for multiplexing within the same reaction volume for a plurality of nucleic acid displacement circuits.

In another aspect, the disclosure provides a system, comprising a nanopore system, a plurality of distinct double stranded nucleic acid complexes, and a computing system communicatively coupled to the nanopore system. The nanopore system comprises:

a nanopore disposed in a barrier defining a cis side and a trans side, wherein the cis side comprises a first conductive liquid medium and the trans side comprises a second conductive liquid medium, and wherein the nanopore comprises a tunnel that provides liquid communication between the cis side and the trans side;

a controllable voltage source connected to the cis side and the trans side by one or more electrodes, wherein the controllable voltage source is configured to generate an electrical potential across the barrier; and

a data acquisition device operable to detect an ion current through the nanopore.

Regarding the plurality of distinct double stranded nucleic acid complexes, each double stranded nucleic acid complex comprises a partner strand hybridized along a first portion of the partner strand to an output strand along a portion of the output strand. The nanopore system is operative to individually translocate the output strands in single stranded format from the first conductive liquid medium toward the second conductive liquid medium through the tunnel and detect an ion current through the nanopore while each output strand is in the tunnel.

The computing system includes logic that, in response to execution by at least one processor of the computing system, causes the computing system to perform actions for analyzing a current pattern from the detected ion current, the actions comprising: receiving from the nanopore system a plurality of signals, wherein the plurality of signals represent an ion current pattern detected in the nanopore while each output strand is in the tunnel; and

providing the plurality of signals, or the one or more signal parameters extracted therefrom, to a machine learning model to determine a unique digital fingerprint corresponding to a unique sequence of the output strand.

In another aspect, the disclosure provides a non-transitory computer-readable medium having computer-executable instructions stored thereon that, in response to execution by one or more processors of a computing system, cause the computing system to perform actions for detecting a result of a nucleic acid-based computation. The actions comprise:

receiving, by the computing system from a nanopore system, ionic current signal data generated while processing a plurality of output strands through at least one nanopore;

detecting, by the computing system, a plurality of capture events within the ionic current signal data;

determining, by the computing system, concentrations of the plurality of output strands based on the plurality of capture events over time; and

providing, by the computing system, the concentrations of the plurality of output strands as an output of the DNA-based computation.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a diagrammatic overview of a catalytic seesaw gate circuit output detection with a nanopore sensor. Circuit components are mixed and loaded into a nanopore sensor array for real-time readout. Input strands react with the amplifier, which displaces the 3′ labeled biotin-streptavidin output ssDNA (Step 1), which is then free to be captured and read by a nanopore sensor. The input strand is recycled (Step 2).

FIG. 1B is an illustrative trace readout of raw ionic current data generated in the nanopore system (i.e., MinION®) over time. Capture events are depicted when the current drops from open pore to a lower level between voltage flips. The lower current level, or “blockade”, occurs when the output strand is captured and held statically in the constriction area of the pore and, thus, is associated with the capture of the output strand in the nanopore. Two successive capture events of the same output strand are illustrated.

FIG. 1C graphically illustrates absolute quantification of output strand concentration. The number of events (after 5 minutes) are plotted against the mean fractional current for buffer (control), 3 μM Strep (control), and 0.5 μM Output DNA+3 μM Strep.

FIG. 1D is a standard curve relating average time between output strand captures to known concentrations of output.

FIG. 1E graphically illustrates comparison of reporter strategies. Kinetic plot as quantified from nanopore raw data are illustrated, showing concentration of output strand over two hours. For comparison, the experiment was replicated on a fluorescence plate reader (PR) using FAM fluorophore as the means of reporting.

FIG. 1F illustrates mapping an output strand's nanopore-sensitive region. The left panel is a cartoon cross section of a nanopore with an output strand held statically in the tunnel by an anchor moiety, with barcode residues numbered. The right panel is a plot showing the change in the median nanopore raw signal elicited by a single-nucleotide mutation at the different numbered positions on the strand's barcode.

FIG. 2A graphically illustrates the fractional mean nanopore output signal for a series of ten DNA strand displacement circuits, each with a unique, pseudo-random barcode sequence in the output strand.

FIG. 2B schematically illustrates the application of a convolutional neural network (CNN) machine learning classifier to signals obtained from the nanopore-based detection of the DNA-circuit to identify the barcode corresponding to the raw nanopore output signals.

FIG. 2C is a confusion matrix demonstrating that the randomly generated barcodes are clearly distinguished from each other using the trained CNN classifier illustrated in FIG. 2B.

FIG. 2D illustrates results of a two-circuit multiplex reaction. The first panel schematically illustrates the reaction set-up wherein distinct displaced output strands from two different DNA circuits are detected in a nanopore array in the same reaction. The raw output signals were subjected to the CNN classifier, which was able to distinguish the barcodes that were present from other barcodes in the random set (second panel and third panel).

FIG. 3A graphically illustrates the comparison of the observed mean fractional current for each of the randomly-generated barcodes versus the model prediction for the barcode sequences.

FIG. 3B graphically illustrates the comparison of the observed mean fractional current for each of the specifically designed barcodes versus the model prediction for the barcode sequences.

FIG. 3C is a confusion matrix demonstrating that the specifically designed barcodes are clearly distinguished from each other using the trained CNN classifier. Barcode number 4 is not included due to a paucity of data.

### DETAILED DESCRIPTION

As indicated above, the specificity and programmability of nucleic acid hybridization interactions offer flexible and fine-tuned control over reacting species. Among the DNA (or more broadly, nucleic acid) computing techniques used today, strand displacement circuits are highly popular, with potential applications ranging from disease diagnostics to nucleic acid-based artificial neural networks. The fundamental mechanism of these circuits is the hybridization of a single-stranded nucleic acid (e.g., DNA) input strand to a double-stranded complex that triggers the release of an output strand. When released, the output strand can be detected and used to characterize circuit behavior. The output strands of strand displacement circuits are typically read out using fluorescence spectroscopy. However, due to spectral overlap of traditional reporters (e.g. FAM, TAMRA, Cy5, and the like), the number of outputs that can be detected in parallel is limited.

This disclosure is based on the inventor's adaptation of nanopore sensing technology as an alternative readout method that facilitates more multiplexed, real-time detection and quantification of nucleic acid (e.g., DNA) strand displacement reactions. As described in more detail below, dynamic sensing of an operating circuit within the flow cell of a commercially-available high-throughput nanopore sensor array (MinION®, Oxford Nanopore Technologies) was demonstrated. Furthermore, it was shown that strand capture frequency can be correlated to concentration, allowing for direct quantification of desired circuit elements. To investigate the multiplexing potential this reporter strategy, a collection of ten orthogonal circuit output sequences (barcodes) were presented into the nanopore system and were able to be classified at the single-molecule level from raw nanopore signal data using machine learning. This demonstrates the potential to scale to much larger barcode sets given the diversity of barcodes that can lead to unique signals in the nanopore system. The research establishes that nanopore-based detection of strand displacement circuits holds key advantages over fluorescence-based methods for real-time, multiplexed circuit readout on an inexpensive, portable sensor device.

In accordance with the foregoing, the disclosure provides a method of detecting a nucleic acid strand displacement circuit in a nanopore system. The nanopore system comprises a nanopore disposed between a first conductive liquid medium and a second conductive liquid medium, wherein the nanopore comprises a tunnel that provides liquid communication between the first conductive liquid medium and the second conductive liquid medium. Illustrative elements and features of nanopores and nanopore systems encompassed by this disclosure are described in more detail below.

The method comprises contacting a double stranded complex with an input strand, wherein the double stranded complex comprises a partner strand hybridized along a first portion of the partner strand to an output strand along a portion of the output strand; permitting hybridization of the input strand to the partner strand along a second portion of the partner strand that partially overlaps with the first portion of the partner strand, thereby displacing the output strand from the double stranded complex; translocating the displaced output strand through the nanopore from the first conductive liquid medium towards the second conductive liquid medium; measuring an ion current through the nanopore when the displaced output strand is in the tunnel to provide a current pattern corresponding to a portion of the displaced output strand; and associating the current pattern with the input strand that displaced the output strand, thereby detecting the nucleic acid strand displacement circuit.

A nucleic acid circuit is a construct that has gained prominence in, e.g., molecular engineering and nucleic acid-based computations systems. It refers to a particular organization of nucleic acid molecules, whereby a repository of nucleic acid constructs (in the present case, double stranded) can be queried by contacting the constructs with an input nucleic acid strand. If the input strand has an appropriate sequence such that it can hybridize with a partner strand in the double stranded complex, it will displace an output strand from the double stranded complex. Detection of the displaced output strand indicates completion of the nucleic acid circuit and serves as an output signal in response to the initial query. The input strand itself can be obtained from biological samples or can be output strands from other nucleic acid displacement circuits. Such nucleic acid circuits can have a variety of applications, depending on the information integrated within the complementary sequences of the component strands.

The input, partner, and output strands, when considered individually, can be or comprise any type of single stranded nucleic acid. The term “nucleic acid” refers to any polymer molecule that comprises multiple nucleotide subunits (i.e., polynucleotides). Nucleic acids encompassed by the present disclosure can include deoxyribonucleotide polymer (DNA), ribonucleotide polymer (RNA), cDNA, or a synthetic nucleic acid known in the art, such as peptide nucleic acid (PNA), glycerol nucleic acid (GNA), threose nucleic acid (TNA), locked nucleic acid (LNA), or other synthetic polymers with nucleotide side chains, or any combination thereof.

Nucleotide subunits of the nucleic acid polymers can be naturally occurring or artificial or modified. A nucleotide typically contains a nucleobase, a sugar, and at least one phosphate group. The nucleobase is typically heterocyclic. Suitable nucleobases include purines and pyrimidines and more specifically adenine (A), guanine (G), thymine (T) (or typically in RNA, uracil (U) instead of thymine (T)), and cytosine (C). The sugar is typically a pentose sugar. Suitable sugars include, but are not limited to, ribose and deoxyribose. The nucleotide is typically a ribonucleotide or deoxyribonucleotide. The nucleotide typically contains a monophosphate, diphosphate, or triphosphate. These are generally referred to herein as nucleotides or nucleotide residues to indicate the subunit. Without specific identification, the general terms nucleotides, nucleotide residues, and the like, are not intended to imply any specific structure or identity. The nucleotides can also be synthetic or modified. For example, the nucleotide can be modified in a manner that provide a distinct signal when in the nanopore tunnel. This can enhance the differentiation of the resulting signal for the targeted residue (subunit) when, e.g., incorporated in a unique sequence, e.g., barcode, sequence of the output strand. For example, see International Application No. PCT/US2014/53754, incorporated herein by reference in its entirety. An exemplary modification for the practice of the present disclosure is to incorporate a nucleic acid residue with a missing base structure, for example, an abasic unit or spacer in the polynucleotide into the output strand. This is particularly advantageous because abasic residues have been observed to result in a marked current spike (i.e., sharp increase in current) when positioned within the constriction zone. Accordingly, the specific position of the abasic residue (or residues) can be readily monitored and their presence are readily identifiable compared to canonical residues. These modified or abasic nucleotide positions offer additional variation to increase theoretical complexity of differentiable barcode sequences.

For any particular nucleic acid circuit, the input strand, the partner strand, and the output strand can independently be or comprise the same or different nucleic acid types. For example, the input strand, the partner strand, and the output strand can all be DNA constructs (i.e., to provide a DNA circuit). In other embodiments, one of the indicated strands can be DNA, whereas one or both of the other strands can be RNA or PNA, so long as the hybridization functionalities are maintained, as described below.

As indicated, in a nucleic acid circuit encompassed by this disclosure, a double stranded complex contains a partner strand and an output strand hybridized thereto. In the resting state (i.e., prior to contacting with an appropriate input strand), the partner strand is hybridized along a first portion of the partner strand to an output strand along a portion of the output strand. The output strand does not hybridize to the entirety of the partner strand. In some embodiments, each member of the double stranded complex, i.e., each of the partner strand and the output strand, has a portion that is not hybridized to the other. For example, the partner strand can comprise a toehold sequence and a partner sequence, whereas the output strand comprises a first domain a second domain. The first domain of the output strand has a sequence that hybridizes to the partner sequence of the partner strand (i.e., the “first portion” of the partner strand). The second domain of the output sequence can have a unique sequence such as a barcode sequence that may or may not also hybridize to a subsequence of the partner sequence. In some embodiments, the barcode sequence does not hybridize with any portion of the partner strand. In other embodiments, the barcode sequence hybridizes (or partially hybridizes) with a corresponding sub-sequence of the partner sequence.

As indicated above, the partner strand contains a defined second portion that partially overlaps with the defined first portion. The subdomain of the second portion that does not overlap can be or comprise a toehold sequence. The toehold sequence is typically close to or contiguous with the partner sequence. As the toehold sequence is not part of the “first portion” of the partner strand, it does not correspond to any sequence in the output strand, which only hybridizes with the first portion of the partner strand. Accordingly, the output strand does not hybridize with the toehold sequence of the partner strand.

The input strand comprises a sequence that hybridizes to the second portion of the partner strand. Specifically, the input strand comprises a sequence that hybridizes to the toehold sequence and at least a portion of the partner sequence of the partner strand. When the input strand comes into contact with the double stranded complex, the toehold sequence is accessible for hybridization to a portion of the input strand. The input strand and the output strand share a commonality of a sequence that will hybridize to the partner sequence of the partner strand. Thus, once hybridized to the toehold sequence, the input strand displaces the output strand from the partner sequence due to competitive hybridization for the partner sequence.

In some embodiments, the partner strand is between about 15 nucleotides and about 150 nucleotides long. In some embodiments, the partner strand is between about 15 and about 100 nucleotides long, for example, between about 15 and about 90, between about 15 and about 80, between about 15 and about 70, between about 15 and about 60, between about 15 and about 50, between about 15 and about 40, between about 15 and about 35, between about 25 and about 90, between about 25 and about 80, between about 25 and about 70, between about 25 and about 60, between about 25 and about 50, between about 25 and about 40, between about 25 and about 35, between about 35 and about 90, between about 35 and about 80, between about 35 and about 70, between about 35 and about 60, between about 35 and about 50, or between about 35 and about 40 nucleotides long. Exemplary, non-limiting partner strand lengths included about 35 nucleotides, about 40 nucleotides, about 45 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, about 110 nucleotides, about 120 nucleotides, about 130 nucleotides, about 140 nucleotides, and about 150 nucleotides.

In some embodiments, the toehold sequence of the partner strand is between about 2 nucleotides and about 20 nucleotides long, although it can be longer. For example, the toehold sequence can be 2, 3, 4, 5, 6, 7, 8, 9, 10, 11, 12, 13, 14, or 15 nucleotides long. In some embodiments, the partner sequence of the partner strand is between about 15 nucleotides and about 50 nucleotides long, such as between about 15 and about 45, between about 15 and about 40, between about 15 and about 35, between about 15 and about 30, between about 15 and about 25, between about 15 and about 20, between about 20 and about 50, between about 20 and about 45, between about 20 and about 40, between about 20 and about 35, between about 20 and about 30, between about 20 and about 25, between about 30 and about 50, between about 30 and about 45, between about 30 and about 40, between about 30 and about 35, or between about 40 and about 50 nucleotides long.

In some embodiments, the input strand is between about 15 nucleotides and about 50 nucleotides long. Similarly, in some embodiments the output strand is between about 15 nucleotides and about 50 nucleotides long. For example, in some embodiments, the input strand and/or the output strand can independently be between about 15 nucleotides and about 50 nucleotides long, such as between about 15 and about 45, between about 15 and about 40, between about 15 and about 35, between about 15 and about 30, between about 15 and about 25, between about 15 and about 20, between about 20 and about 50, between about 20 and about 45, between about 20 and about 40, between about 20 and about 35, between about 20 and about 30, between about 20 and about 25, between about 30 and about 50, between about 30 and about 45, between about 30 and about 40, between about 30 and about 35, between about 35 and about 50, between about 35 and about 45, between about 35 and about 40, or between about 40 and about 50 nucleotides long.

The output strand has a unique sequence in its second domain such that when the output strand is displaced from the double stranded complex by the input strand, the displaced output strand can be captured by, and translocated through, the nanopore to provide a detectable signal that is sufficiently unique to identify the output strand and infer its displacement by an input strand, thus permitting detection of the nucleic acid circuit.

Exemplary, non-limiting partner strands (referred to as “Gate”) and corresponding output and input strands are set forth in Table 1 below. The exemplary partner strands sequences are set forth herein as SEQ ID NOS:1, 5, 9, 13, 17, 21, 25, 29, 33, and 37. The corresponding exemplary output strands sequences are set forth herein as SEQ ID NOS:2, 6, 10, 14, 18, 22, 26, 30, 34, and 38. The barcode regions of these exemplary output strands are indicated with “ ” as the implemented barcodes are not limited and can theoretically be any sequence. The corresponding exemplary input strands sequences are set forth herein as SEQ ID NOS:3, 7, 11, 15, 19, 23, 27, 31, 35, and 39.

In some embodiments, the unique sequence is a barcode sequence. The barcode sequence can be randomly generated or rationally designed to produce a distinct current signal in a particular nanopore system platform. The length of the barcode can depend on dimensions and sensitivity of the selected nanopore system. Typical lengths can be from about 2 to about 20 nucleotides, from about 2 to about 15 nucleotides, from about 2 to about 20 nucleotides, from about 3 to about 9 nucleotides, from about 4 to about 8 nucleotides and from about 4 to about 7 nucleotides long. For example, in some embodiments, the barcode length can be about 2, 3, 4, 5, 6, 7, 8, 9, 10, 11, 12, 13, 14, or 15 nucleotides long.

In the exemplary embodiments set forth in Table 1, the output strands have a 6 nucleotide barcode sequence. The exemplary barcodes implemented as a proof of concept into the disclosed output strand sequences are set forth in Table 2, which provides a set of semi-random barcodes that are distinct from each other. Table 2 also provides a set of rationally design barcode sequences based on the known performance parameters of the specific nanopore system. All of the disclosed partner strand, output strand, input strand, and exemplary barcode sequences are encompassed by the present disclosure. However, these are merely illustrative and a person of ordinary skill in the art will readily be able to design alternative examples that are encompassed by the present disclosure.

In some embodiments, the output strand also comprises an anchor moiety. The anchor moiety can be incorporated into the output strand prior to, during, or after displacement from the double stranded complex. The anchor moiety has dimensions that exceed the diameter of the nanopore tunnel, thus preventing, inhibiting, or slowing passage through the nanopore.

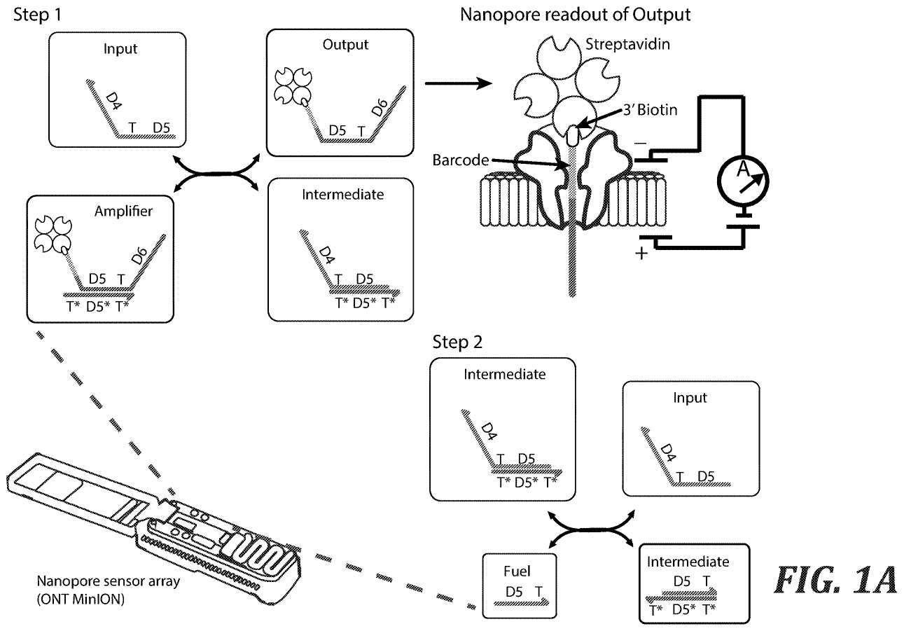

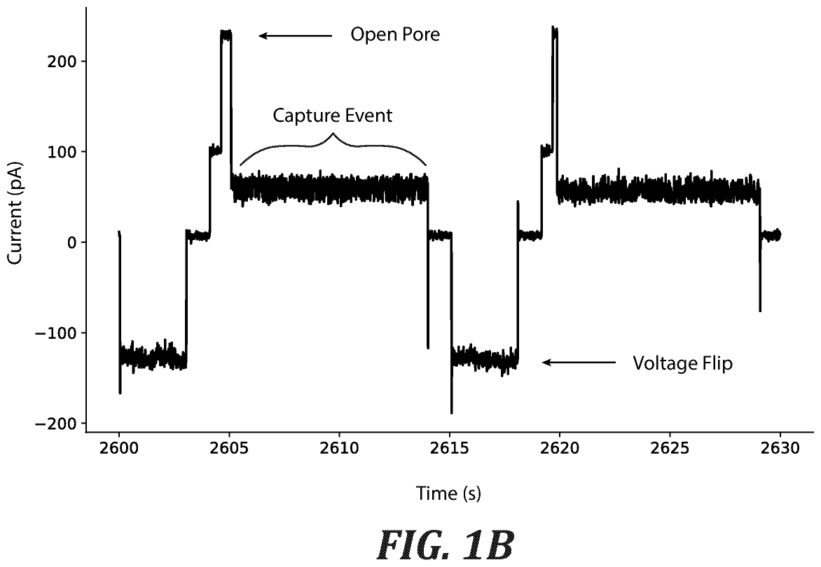

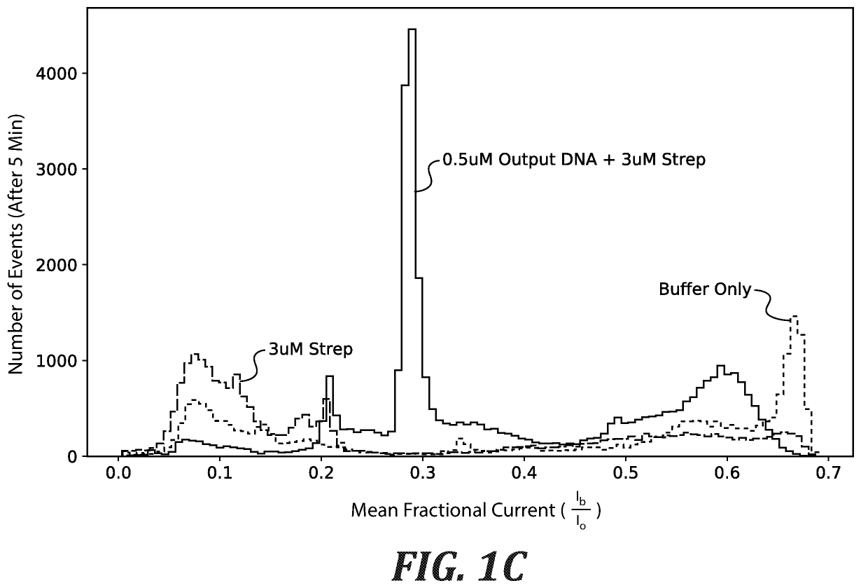

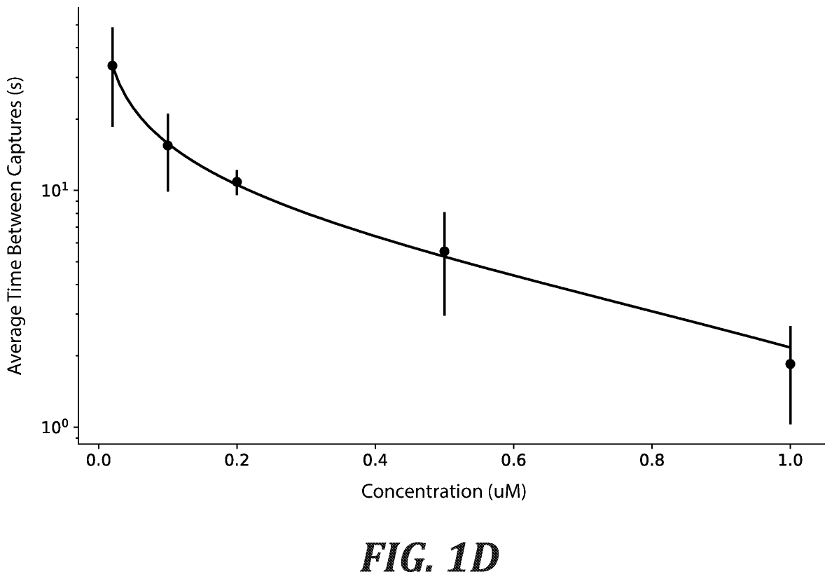

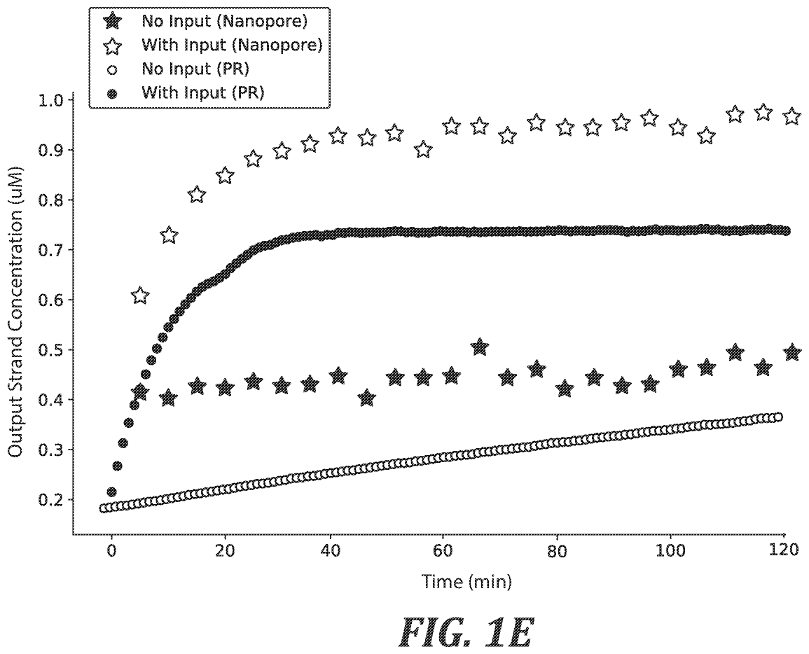

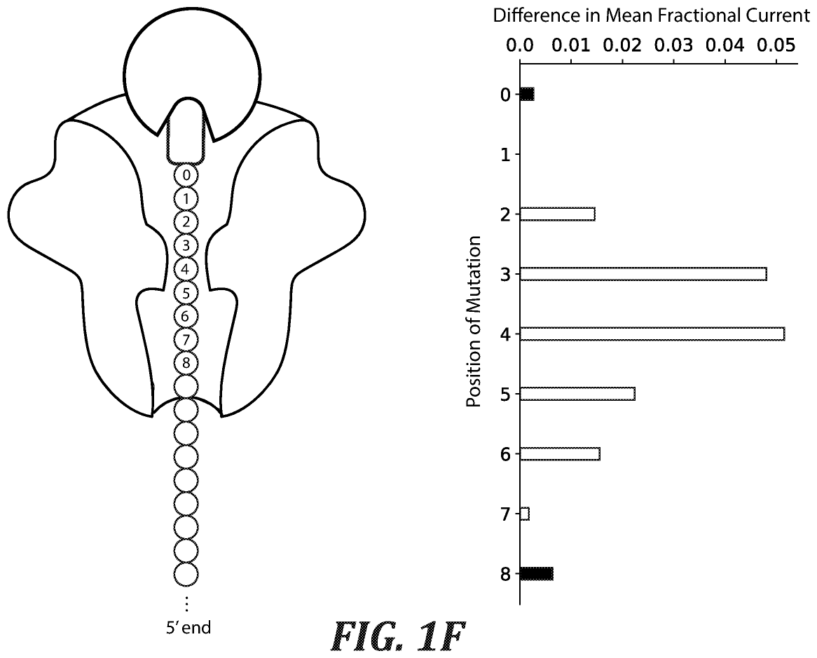

In some embodiments, the anchor moiety is configured inhibit or slow translocation when the anchor moiety contacts an outer surface of the nanopore or an inner constriction region of the nanopore. Due to the dimensions of the anchor moiety, it will not readily pass through the nanopore tunnel, thus slowing the rate of translocation of the attached output strand towards the second conductive liquid medium on the trans side of the nanopore. This reduced translocation rate results in increased residence time of the barcode sequence in the nanopore tunnel to facilitate recordation of a current signal that is representative of the barcode sequence. In some embodiments, the anchor moiety is configured to arrest translocation of the displaced output strand when the anchor moiety contacts an outer surface of the nanopore. This results in the barcode sequence of the displaced output strand being held statically within the tunnel of the nanopore when the ion current is measured (see FIG. 1F, left panel). The position of the anchor moiety relative to the barcode will depend on the dimensions of the nanopore used such that upon slowing or arrest of translocation, the barcode sequence is appropriately positions to affect current signal. In some embodiments, the anchor moiety is at the 3′ terminal end of the output strand. This is typically the case if the nanopore tends to capture the 5′ end to initiate translocation. In other embodiments, the anchor moiety is at the 5′ terminal end of the output strand. This is typically the case if the nanopore tends to capture the 3′ end to initiate translocation. In other embodiments, the anchor moiety is attached to or spans part of an internal nucleic acid or acids in the output strand. A person of ordinary skill in the art can determine the correct position of the anchor moiety to ensure that the slowed or arrested translocation results in the extended residence of the barcode sequence within the constriction zone of the nanopore tunnel such that the resultant signal is reflective of the barcode sequence structure and not another aspect of the output strand sequence. See, e.g., FIG. 1F and the related description in Example 1 below, which addresses an illustrative example of properly positioning the anchor moiety relative to the barcode in a nanopore system to maximize the barcode influence on the resultant signal.

In one embodiment, the anchor moiety comprises biotin. Biotin can be conjugated to the output strand according to known techniques at a rationally chosen position designed to strategically pause or arrest translocation when the barcode sequence is in the constriction zone of the nanopore tunnel, as described above. For example, in the embodiment described in more detail in Example 1, the biotin is conjugated at the 3′ terminal end of the output strand. The biotin can be conjugated to the output strand prior to the output strand being integrated into the double stranded complex with the partner strand. In further embodiments, the anchor moiety can comprise a biotin-binding partner, such as avidin, neutravidin, or streptavidin, that is conjugated to the biotin. Biotin-binding partners, such as avidin, neutravidin, or streptavidin, bind to biotin noncovalently with very high affinity and specificity. Due to their size, the biotin-binding partners are useful components of the anchor moiety to arrest translocation of the output strand in the nanopore. Furthermore, due to the specificity of the biotin-binding partners for biotin, the biotin-binding partner can be incorporated into the output strand at any convenient time prior to the capture and translocation of a displaced output strand through the nanopore. For example, the biotin-binding partner can be integrated into the output strand (by virtue of the non-covalent bond to biotin) before the output strand is integrated into the double stranded complex. In other embodiments, the biotin-binding partner can be integrated into the output strand after the double stranded complex is formed. In further embodiments, the double stranded complex biotin-binding partner can be integrated into the output strand before, during, or after the input strand displaces the output strand from the double stranded complex.

While the present description has focused primarily on the use of biotin and biotin-binding partners as the anchor moiety, the disclosure is not limited thereto. The present disclosure encompasses any anchor moiety that can be specifically integrated into the output strand in a manner configured to slow or pause translocation while the barcode sequence is appropriately positioned within the nanopore tunnel to produce a unique signal. Other exemplary anchor moieties that can be readily integrated into the output strand include proteins or peptides with secondary or tertiary structure providing stable dimensions that exceed the diameter of the tunnel and does not pass through the nanopore. Yet further examples include nucleic acid moieties with secondary structure, such as DNA hairpins, G-quadruplex, and the like. Exemplary disclosures addressing use of DNA hairpins and other arresting constructs that can be used as anchor moieties are described in, e.g., WO 2014/022800, WO 2011/106459, and Manrao et al., “Reading DNA at single-nucleotide resolution with a mutant MspA nanopore and phi29 DNA polymerase,” Nature Biotechnology 30:349-353 (2012), each of which is incorporated herein by reference in its entirety.

The steps of the method can be performed together in a single reaction volume or separately. For example, the contacting and hybridization steps with the input strand can be performed in the first conductive liquid medium of the nanopore system. Thus, any displaced output strands can be permitted to interact and translocate through the nanopore for detection without further transfer. Alternatively, the contacting and hybridization steps with the input strand can be performed in one reaction volume separate from the nanopore system and thereafter any displaced output strands can be transferred to the nanopore system for detection (i.e., the translocating, measuring, and associating steps).

As described in more detail below, the nanopore based methods can incorporate use of applied electrical potentials between the first conductive liquid medium and the second conductive liquid medium to promote translocation of the displaced output strand through the nanopore towards the second conductive liquid medium. Furthermore, in some embodiments, the polarity of the electrical potential is reversed after a predetermined time to clear the nanopore. This is applicable when the anchor moiety is configured to retain its position on the output strand so as to arrest translocation regardless of the initially applied electrical potential. The reversed polarity is sufficient to reverse translocation direction towards the first conductive liquid medium, thereby causing the displaced output strand to exit the nanopore into the first conductive liquid medium leaving the nanopore open to potentially capture another output strand for analysis and detection. Such a reversal, or “voltage flip,” is reflected in the exemplary current output pattern shown in FIG. 1B, where the nanopore is cleared after a period of time and can capture and analyze a subsequent output strand. The predetermined amount of time before a voltage flip is typically a time sufficient to measure an ion current that is reflective of the barcode sequence structure that resides in the nanopore during the arrested translocation.

In other embodiments wherein the anchor moiety is configured to release displaced output strand, or otherwise merely slow translocation, the nanopore can be cleared by allowing translocation to continue from the first conductive liquid medium ultimately into the second conductive liquid medium on the trans side. In this embodiment, the polarity of the initially applied electrical potential is not reversed, but rather is maintained for a sufficient time to complete translocation.

The method has heretofore been described in the context of a single input strand displacing a single output strand from a single double stranded complex. However, it will be understood that in practice the method can be conducted in the context of a plurality of each circuit component (i.e., a plurality of the same input strand and a plurality of the same double stranded complex) in a common reaction volume. In such scaled-up reactions, the method can further comprise repeating the steps of translocation, measuring the ion current, and clearing the nanopore (e.g., by reversing polarity) one or more times. This permits quantification of capture events over time, where each capture event is identified by the same current pattern that corresponds to the particular (e.g., barcode) sequence of the displaced output strand. The measurements of rate of capture over a defined time period can be associated with the amount, or concentration, of single stranded (i.e., displaced) output strands within the reaction volume. The associations can be made, e.g., by comparison to a standard curve showing capture rate of output strands according concentration in the reaction volume for that particular nanopore. Due to the 1:1 relationship of competitive hybridization, the concentration of displaced output strands indicates the concentration of the input strands that are successfully able to displace the output strands. In some embodiments, the time between capture events can be correlated with the concentration of the associated input strand in the common reaction volume. In other embodiments, a rate of capture events, e.g., the number of capture events for a defined period of time, is correlated with the concentration of the associated input strand in the common reaction volume. In these scenarios, it will be understood that the multiple capture events are determined to be of the same unique (e.g., barcode) sequence indicating the same type of displaced output strand.

As indicated above and described in more detail below, the disclosed method provides the capability to multiplex by allowing distinction of a large number of different barcode sequences and, therefore, for detection and quantification of a large number of distinct nucleic acid circuits. The degree of multiplexing is theoretically limited only by the capacity of the nanopore system to differentiate the variety of available barcode signals, which is vast. As described in more detail below, it is established that machine learning models can be readily taught to successfully differentiate signals appear to group together based on a few extracted parameters of the current signal. Thus, in one embodiment, the method comprises performing the method for a plurality of distinct nucleic acid strand displacement circuits in a common reaction volume. In such multiplexed reactions, the common reaction volume comprises a plurality of distinct double stranded complexes with distinct sequences in their respective second portion of the partner strand. The plurality of distinct double stranded complexes can serve as a molecular database that can receive multiple queries in the form of distinct input strands. The output strand of each distinct double stranded complex comprises a unique barcode sequence that, if detected, completes the strand displacement circuit and indicates the presence of the input strand. As indicated above, the detected strand displacement circuit(s) can also be quantified, as described above, providing additional information.

The current pattern obtained from measuring the ion current through the nanopore is ideally uniquely associated with a single barcode sequence. For example, the nanopore system may have the sensitivity to ascertain the primary sequence of the barcode sequence. However, such resolution of signal is not necessary. It is sufficient to simply recognize that a particular barcode sequence produces a unique current pattern in the nanopore system that is recognizable and differentiable from current patterns produced by other barcode sequences. In this sense, the current pattern reflects a unique “fingerprint.” In this regard, the term “fingerprint” is used to refer to sufficient structural or sequence data that can be used to determine whether two sequences (e.g., barcode sequences) are different or whether they are likely the same based on the current pattern.

In some embodiments, the association can be formed by referring to a database of current patterns for established barcodes within a particular nanopore system. Such databases may be pre-existing or generated specifically for a nanopore system configuration. In some embodiments, the association is performed by a computing system. For example, the step of associating the current pattern with the input strand to detect the one or more nucleic acid strand displacement circuits can comprise providing the current pattern, or one or more signal parameters extracted from the current pattern, to a machine learning model to determine a unique digital fingerprint. The term “unique digital fingerprint” refers to one or more characteristics established from the current pattern (or signal parameters extracted therefrom) that establish the presence of an identifiable discrete barcode species.

In some embodiments, one or more signal parameters can be manually extracted from the current pattern of each capture event and then input into the machine learning model. Such machine learning models can include non-neural network models, such as Support Vector Machine, Random Forest, and the like. In other embodiments, the machine learning models are capable of extracting the pertinent one or more signal parameters directly from the current pattern. Such machine learning models can include neural network machine learning models, such as a convolutional neural network (CNN). Exemplary signal parameters that have been demonstrated to be informative for the machine learning models include mean current, median current, minimum current, maximum current, and/or standard deviation of current. Such signal parameters can be used in any combination to establish the unique digital fingerprint. The unique digital fingerprint is then used to identify and differentiate specific barcodes that are detected. The detection of the displacement nucleic acid circuit element is correlated with the input strand that led to the displacement of the output strand.

As indicated above, the steps of the multiplexed reactions can be repeated multiple times. For example, the nanopore can be cleared by reversing polarity of an electrical potential applied during translocation to cause the displaced output strand(s) to exit the nanopore(s) into the first conductive liquid medium. The method steps of translocation, measuring ion current, and reversing polarity are repeated one or more times. Capture events are quantified of over time, e.g., by measuring the rate of capture or time between capture events for any one or more specific displacement circuits, as determined by the one or more unique digital fingerprints.

The detection (and potentially quantification) of one or more nucleic acid strand displacement circuits using nanopore-based detection can be employed in a variety of applications, accordingly to the knowledge and expertise of a person of ordinary skill in the art. To illustrate, the disclosed method can be a molecular diagnostic method wherein a query sample may contain one or more nucleic acid strands obtained from a subject that are biomarkers of disease(s). The reaction solution can contain a library of double stranded complexes, each containing sequence that is associated with a particular disease. This library can be interrogated by contacting the reaction solution with the query sample. The potential biomarker strands can serve as the input strands in the above method, and the presence of any relevant biomarker strands in the query sample can be determined by detection of one or more nucleic acid strand displacement circuits associate with the one or more biomarker sequences.

Alternatively, the described method can be integrated into methods of molecular—(i.e., nucleic acid-) based computational systems, such as amplifiers (see, e.g., Zhang, David Yu, et al., “Engineering entropy-driven reactions and networks catalyzed by DNA.” Science 318(5853):1121-1125 (2007)), Boolean logic gates (Qian, Lulu, and Erik Winfree, “Scaling up digital circuit computation with DNA strand displacement cascades.” Science 332(6034):1196-1201 (2011), Seelig, Georg, et al., “Enzyme-free nucleic acid logic circuits.” science 314(5805):1585-1588 (2006), and Cherry KM and Qian L, “Scaling up molecular pattern recognition with DNA-based winner-take-all neural networks,” Nature 559:370-376 (2018)), chemical reaction networks (Chen, Yuan-Jyue, et al., “Programmable chemical controllers made from DNA.” Nature nanotechnology 8(10):755-762 (2013)), oscillators (Srinivas, Niranjan, et al., “Enzyme-free nucleic acid dynamical systems.” Science 358(6369) (2017)), and neural networks (Qian, Lulu, et al., “Neural network computation with DNA strand displacement cascades.” Nature 475(7356): 368-372 (2011), Cherry KM and Qian L, Nature 559:370-376 (2018), supra). Each reference is incorporated herein by reference in its entirety.

Nanopores and Nanopores Systems

Various aspects of the nanopores and nanopore systems as employed in the disclosed methods and systems are described here.

Nanopore-based analysis methods have previously been investigated for the characterization of analytes that are passed through the nanopore. For example, nanopore systems have been established specifically for the analysis of nucleic acid polymers, for example single-stranded DNA (“ssDNA”) and single-stranded RNA, which pass linearly through a nanoscopic opening of the nanopore. As the nucleic acid resides in, or moves through (i.e., translocates), the interior of the pore, it modulates the ionic current that passes through the pore depending on the physical characteristics of the nucleic acid molecule. Nanopores have sufficiently constricted tunnels that the ion current is influenced by the sequence structure of the nucleic acid. Thus, the nanopore system provides a signal, such as an electrical signal (e.g., measured current level), that is influenced by the physical properties of the nucleotide subunits that reside in the close physical space of the nanopore tunnel at any given time. The output signal, such as a current level, can be determined by a single, monomeric subunit of the polymer residing in the pore at each iterative translocation step. Thus, as the polymer translocates the resulting trace of output signals can be translated directly into the primary sequence of the polymer. However, most nanopores have constriction zones that physically interact with more than one polymer subunit during any single translocation event and, thus, the magnitude of the ion current is influenced by more than one subunit (e.g., by an “n-mer”). Accordingly, the resulting output signal reflects a plurality of contiguous subunits. See, e.g., FIG. 1F. However, each output signal can still be correlated to signals produced by known combinations of polymer subunits.

The nanopore optimally has a size or three-dimensional configuration that allows the output nucleic acid strand to pass through only in a sequential, single file order. Chemical and physical properties of each monomeric nucleic acid subunit of the output strand can influence electrical signals. Thus, the particular sequence, such as a barcode sequence, can result in a detectable signal characteristic of the output strand (e.g., via a unique barcode) as it passes through and/or resides within nanopore.

A “nanopore” specifically refers to a pore typically having a size of the order of a few nanometers that allows the passage of analyte polymers (such as nucleic acid polymers) therethrough. Typically, nanopores encompassed by the present disclosure have an opening with a diameter at its most narrow point of about 0.3 nm to about 2 nm. Nanopores useful in the present disclosure include any pore capable of permitting the linear translocation of the nucleic acid output strand.

Nanopores can be biological nanopores (e.g., proteinaceous nanopores), solid state nanopores, hybrid solid state protein nanopores, a biologically adapted solid state nanopore, a DNA origami nanopore, and the like.

In some embodiments, the nanopore comprises a protein, such as alpha-hemolysin, anthrax toxin and leukocidins, and outer membrane proteins/porins of bacteria such as Mycobacterium smegmatis porins (Msp), including MspA, outer membrane porins such as OmpF, OmpG, OmpATb, and the like, outer membrane phospholipase A and Neisseria autotransporter lipoprotein (NaIP), and lysenin, as described in U.S. Publication No. US2012/0055792, International PCT Publication Nos. WO2011/106459, WO2011/106456, WO2013/153359, and Manrao et al., “Reading DNA at single-nucleotide resolution with a mutant MspA nanopore and phi29 DNA polymerase,” Nat. Biotechnol. 30:349-353 (2012), each of which is incorporated herein by reference in its entirety. In other embodiments the protein nanopore is CsgG, ClyA, or aerolysin. Nanopores can also include alpha-helix bundle pores that comprise a barrel or channel that is formed from a-helices. Suitable α-helix bundle pores include, but are not limited to, inner membrane proteins and outer membrane proteins, such as WZA and ClyA toxin. In one embodiment, the protein nanopore is a heteroligomeric cationic selective channel from Nocardia faricinica formed by NfpA and NfpB subunits.

The nanopore can also be a homolog or derivative of any nanopore described above. A “homolog,” as used herein, is a protein from another species that has a similar structure and evolutionary origin. By way of an example, homologs of wild-type MspA, such as MppA, PorM1, PorM2, and Mmcs4296, can serve as the nanopore in the disclosed system. Protein nanopores have the advantage that, as biomolecules, they self-assemble and are essentially identical to one another.

In some embodiments, the nanopore, or the portion thereof in contact with the first conductive liquid medium, has a net neutral or net positive charge.

In addition, it is possible to genetically engineer protein nanopores, thus creating a “derivative” of a nanopore that possesses various attributes. Such derivatives can result from substituting amino acid residues for amino acids with different charges. Thus, the protein nanopores can be wild-type or can be modified to contain at least one amino acid substitution, deletion, or addition. In some embodiments, the at least one amino acid substitution, deletion, or addition results in removal of a steric barrier to translocation of the flexible domains through the nanopore. In some embodiments, the at least one amino acid substitution, deletion, or addition results in a different net charge of the nanopore. In some embodiments, the difference in net charge increases the difference of net charge as compared to the charge exhibited by the displaced output strand to facilitate interaction of the output strand with, and capture by, the nanopore. For example, DNA and RNA have a net negative charge due to the phosphate groups in the backbone. Thus, in some embodiments, the nanopore can contain one or more modifications (e.g., substitutions, additions, or deletions) that either remove negative charges or incorporate neutral or positive charges to enhance the electrostatic attraction between the nanopore and displaced output strands.

In some embodiments, the nanopores can include or comprise DNA-based structures, such as generated by DNA origami techniques. For descriptions of DNA origami-based nanopores for analyte detection, see PCT Publication No. WO2013/083983, incorporated herein by reference.

Some nanopores can comprise a variably shaped tunnel component through which the output strand can pass. FIG. 1A provides a cartoon diagram that illustrates and exemplary nanopore configuration where the nanopore is disposed in a membrane. As illustrated in this example, the nanopore has an outer entrance rim region in the cis side that provides a relatively wide opening into the tunnel through which the output strand has passed. The anchor moiety (e.g., attached streptavidin) is too large to enter the nanopore tunnel and, thus, is held against the outer rim within the first conductive liquid medium in the cis side. The widest interior section of the tunnel is often referred to as the vestibule. In contrast, the narrowest portion of the interior tunnel is referred to as the constriction zone. The vestibule and a constriction zone together form the tunnel. In the illustrated nanopore the rim and vestibule together form a cone-shaped portion of the interior of the nanopore whose diameter generally decreases from one end to the other along a central axis, where the narrowest portion of the vestibule is connected to the constriction zone. The output strand is held static in the constriction zone during signal acquisition (see also FIG. 1F). Stated otherwise, the vestibule of the illustrated nanopore can generally be visualized as “goblet-shaped.” Because the vestibule is goblet-shaped, the diameter changes along the path of a central axis, where the diameter is larger at one end than the opposite end. The diameter may range from about 2 nm to about 6 nm. Optionally, the diameter is about, at least about, or at most about 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 nm, or any range derivable therein. The length of the central axis may range from about 2 nm to about 6 nm. Optionally, the length is about, at least about, or at most about 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 nm, or any range derivable therein. When referring to “diameter” herein, one can determine a diameter by measuring center-to-center distances or atomic surface-to-surface distances.

The term “constriction zone” generally refers to the narrowest portion of the tunnel of the nanopore, in terms of diameter, that is connected to the vestibule. The length of the constriction zone can range, for example, from about 0.3 nm to about 20 nm. Optionally, the length is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. The diameter of the constriction zone can range from about 0.3 nm to about 5 nm. Optionally, the diameter is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. In other embodiments, such as those incorporating solid state pores, the range of dimension (length or diameter) can extend up to about 20 nm. For example, the constriction zone of a solid state nanopore is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, or 5 nm, or any range derivable therein. The constriction zone is generally the part of the nanopore structure where the presence of a polymer analyte, such as the output strand, can influence the ionic current from one side of the nanopore to the other side of the nanopore. In some instances, the term “constriction zone” is used in a functional context based on the obtained resolution of the nanopore and, thus, the term is not necessarily limited by any specific parameter of physical dimension. Depending on physical characteristics the nanopore and the overall system, the length (i.e., number of nucleic acid residues in a linear sequence of the output strand) that influences a detectable and distinguishable signal from a nanopore system can vary and be readily determined by a person of ordinary skill in the art for the particular nanopore platform.

In some embodiments, the nanopore can be a solid state nanopore. A solid-state layer is not of biological origin. In other words, a solid-state layer is not derived from or isolated from a biological environment such as an organism or cell, or a synthetically manufactured version of a biologically available structure. Solid state nanopores can be produced as described in U.S. Pat. Nos. 7,258,838 and 7,504,058, incorporated herein by reference in their entireties. Briefly, solid state layers can be formed from both organic and inorganic materials including, but not limited to, microelectronic materials, insulating materials such as Si3N4, Al2O3, and SiO, organic and inorganic polymers such as polyamide, plastics such as Teflon®, or elastomers such as two-component addition-cure silicone rubber, and glasses. The solid-state layer may be formed from graphene. Suitable graphene layers are disclosed in WO 20091035647 and WO 20111046706. Solid state nanopores have the advantage that they are more robust and stable. Furthermore, solid state nanopores can in some cases be multiplexed and batch fabricated in an efficient and cost-effective manner. Finally, they might be combined with micro-electronic fabrication technology. In some embodiments, the nanopore comprises a hybrid protein/solid state nanopore in which a nanopore protein is incorporated into a solid state nanopore. In some embodiments, the nanopore is a biologically adapted solid-state pore.

As indicated, in some embodiments, the nanopore is disposed within a membrane, thin film, layer, or bilayer. For example, biological (e.g., proteinaceous) nanopores can be inserted into an amphiphilic layer such as a biological membrane, for example, a lipid bilayer. An amphiphilic layer is a layer formed from amphiphilic molecules, such as phospholipids, which have both hydrophilic and lipophilic properties. The amphiphilic layer can be a monolayer or a bilayer. In some embodiments, the membrane is an artificial membrane that comprises mycolic acid, which confers additional stability and supports some nanopore configurations additional stability and supports some nanopore configurations (e.g., nanopores derived from mycobacteria). See, e.g., WO 2011/106456, incorporated herein by reference in its entirety. The amphiphilic layer may be a co-block polymer. Alternatively, a biological pore may be inserted into a solid-state layer.

The membrane, thin film, layer, or bilayer typically separates a first conductive liquid medium and a second conductive liquid medium to provide a nonconductive barrier between the first conductive liquid medium and the second conductive liquid medium. The nanopore, thus, provides liquid communication between the first and second conductive liquid media through its internal tunnel. In some embodiments, the pore provides the only liquid communication between the first and second conductive liquid media. The conductive liquid media typically comprises electrolytes or ions that can flow from the first conductive liquid medium to the second conductive liquid medium through the interior of the nanopore. Liquids employable in methods described herein are well-known in the art. Descriptions and examples of such media, including conductive liquid media, are provided in U.S. Pat. No. 7,189,503, for example, which is incorporated herein by reference in its entirety. The first and second liquid media may be the same or different, and either one or both may comprise one or more of a salt, a detergent, or a buffer. Indeed, any liquid media described herein may comprise one or more of a salt, a detergent, or a buffer. Additionally, any liquid medium described herein may comprise a viscosity altering substance or a velocity altering substance.

In some embodiments, the nanopore system can comprise a plurality of nanopores, either all of the same type or of differing types, to facilitate assessment of a plurality of output strands simultaneously. The plurality of nanopores can be organized, for example, in an array, where each nanopore is operable to translocate the displaced output strand from a first conductive liquid medium towards the second conductive liquid medium and to measure an ion current pattern when the displaced output strand is in the tunnel.

In some cases, the first and second conductive liquid media located on either side of the nanopore are referred to as being on the cis and trans regions or sides, where the elements of the potential nucleic acid circuit, including the input strand, the complex with the partner and output strands, are provided in the cis region. In some embodiments, the nanopore or portion thereof in contact with the first conductive liquid medium in the cis region, has a net neutral charge or net positive charge.

Nanopore systems also incorporate structural elements to measure and/or apply an electrical potential across the nanopore-bearing membrane or film. For example, the system can include a pair or series of drive electrodes that drive current through the nanopores. Typically, the negative pole is disposed in the cis region and the positive pole is disposed in the trans region. Additionally, the nanopore system can include a data acquisition device configured to measure the ion current through the nanopore. In some embodiments, the data acquisition device comprises one or more measurement electrodes that measure the current through the nanopore. These can include, for example, a patch-clamp amplifier or a data acquisition device. For example, nanopore systems can include an Axopatch-200B patch-clamp amplifier (Axon Instruments, Union City, Calif.) to apply voltage across the bilayer and measure the ionic current flowing through the nanopore. For example, in some embodiments, the applied electrical field includes a direct or constant current that is between about 10 mV and about 1 V. In some embodiments that include protein-based nanopores embedded in lipid membranes, the applied current includes a direct or constant current that is between about 10 mV and 300 mV, such as about 10 mV, 20 mV, 30 mV, 40 mV, 50 mV, 60 mV, 70 mV, 80 mV, 90 mV, 100 mV, 110 mV, 120 mV, 130 mV, 140 mV, 150 mV, 160 mV, 170 mV, 180 mV, 190 mV, 200 mV, 210 mV, 220 mV, 230 mV, 240 mV, 250 mV, 260 mV, 270 mV, 280 mV, 290 mV, 300 mV, or any voltage therein. In some embodiments, the applied electrical field is between about 40 mV and about 200 mV. In some embodiments, the applied electrical field includes a direct or constant current that is between about 100 mV and about 200 mV. In some embodiments, the applied electrical direct or constant current field is about 180 mV. In other embodiments where solid state nanopores are used, the applied direct or constant current electrical field can be in a similar range as described, up to as high as 1 V. As will be understood, the voltage range that can be used can depend on the type of nanopore system being used and the desired effect.

Persons of skill in the art will readily appreciate that the nanopore system can be configured to reverse electrical potential to the values and ranges described above.

In some embodiments, the electrical potential is not constant, but rather is variable about a reference potential.

An exemplary nanopore system is the MinION® device by Oxford Nanopore Technologies, which was used in the proof of concept experiments described in Example 1.

System

In another aspect, the disclosure provides a system that comprises a nanopore system, a plurality of distinct double stranded nucleic acid complexes, and a computing system communicatively coupled to the nanopore system.

Exemplary elements of a nanopore system, including the nanopores, their dimensions, functionalities, their barriers, etc., are described above and are encompassed in this aspect of the disclosure. The nanopore system of this aspect typically comprises a nanopore disposed in a barrier defining a cis side and a trans side, wherein the cis side comprises a first conductive liquid medium and the trans side comprises a second conductive liquid medium, and wherein the nanopore comprises a tunnel that provides liquid communication between the cis side and the trans side. The nanopore systems also comprise a controllable voltage source connected to the cis side and the trans side by one or more electrodes, wherein the controllable voltage source is configured to generate an electrical potential across the barrier, and a data acquisition device operable to detect an ion current through the nanopore. Illustrative optional elements and features of the nanopore system are described in more detail above.

As indicated, the system also comprises a plurality of distinct double stranded nucleic acid complexes. Each double stranded nucleic acid complex comprises a partner strand hybridized along a first portion of the partner strand to an output strand along a portion of the output strand. The nanopore system is operative or otherwise configured to individually translocate the output strands in single stranded format from the first conductive liquid medium toward the second conductive liquid medium through the tunnel and detect an ion current through the nanopore while each output strand is in the tunnel.

The system also comprises a computing system communicatively coupled to the nanopore system. The computing system including logic that, in response to execution by at least one processor of the computing system, causes the computing system to perform actions for analyzing a current pattern from the detected ion current. The actions comprise:

receiving from the nanopore system a plurality of signals, wherein the plurality of signals represent an ion current pattern detected in the nanopore while each output strand is in the tunnel; and

providing the plurality of signals, or the one or more signal parameters extracted therefrom, to a machine learning model to determine a unique digital fingerprint corresponding to a unique sequence of the output strand.

Features and elements of the double stranded nucleic acid complexes for certain embodiments of this aspect are described in more detail above in the context of the method aspect and are fully encompassed by this aspect of the disclosure.

In some embodiments, the partner strand comprises a toehold sequence and a partner sequence. The output strand comprises a first domain with a sequence that hybridizes to the partner sequence and a second domain with a barcode sequence. In some embodiments, the barcode sequence does not hybridize with any portion of the partner strand. In some embodiments, the partner strand and the output strand of each of the plurality of distinct double stranded nucleic acid complexes are configured to dissociate when an input strand, having a sequence that hybridizes to the toehold sequence and at least a portion of the partner sequence of the partner strand, contacts the double stranded nucleic acid complex.

In some embodiments, the output strand has, or is configured to receive, an anchor moiety, wherein the anchor moiety has dimensions that exceed the diameter of the tunnel preventing passage through the nanopore. The anchor moiety is configured to arrest translocation of the output strand in single strand format when the anchor moiety contacts an outer surface of the nanopore, whereby the barcode sequence of the output strand in single strand format is held statically within the tunnel of the nanopore to permit detection of the ion current. Exemplary, non-limiting elements and embodiments of the anchor moiety are described in more detail above. In a specific embodiment, the anchor moiety comprises biotin. The biotin-based anchor moiety can further comprise biotin-binding partner, such as avidin, neutravidin, or streptavidin conjugated to the biotin. In some embodiments, the biotin-binding partner can be added to the system at a point prior to capture of the output strand by the nanopore.

Each of the plurality of distinct double stranded nucleic acid complexes have a barcode. The barcode sequence is selected from a plurality of distinct barcode sequences. In some embodiments, the distinct barcode sequences of the plurality of distinct barcode sequences can be randomly determined. In other embodiments, the distinct barcode sequences can be selected to have raw nanopore signals that are distinguishable from each other using a machine learning model.

The one or more signal parameters of the current pattern include mean current, median current, minimum current, maximum current, and/or standard deviation of current, in any combination.

Non-Transitory Computer-Readable Medium

In another aspect, the disclosure provides a non-transitory computer-readable medium having computer-executable instructions stored thereon that, in response to execution by one or more processors of a computing system, cause the computing system to perform actions for detecting a result of a nucleic acid-based computation. The actions comprise:

receiving, by the computing system from a nanopore system, ionic current signal data generated while processing a plurality of output strands through at least one nanopore;

detecting, by the computing system, a plurality of capture events within the ionic current signal data;

determining, by the computing system, concentrations of the plurality of output strands based on the plurality of capture events over time; and

providing, by the computing system, the concentrations of the plurality of output strands as an output of the DNA-based computation.

In some embodiments, the action of detecting capture events includes: determining a set of features based on the ionic current signal data; and detecting a capture event in response, at least in part, to determining that the features of the set of features are within an expected range for each feature.

In some embodiments, the action of determining the set of features based on the ionic current signal data includes determining one or more of a mean of the ionic current data, a median of the ionic current data, a minimum of the ionic current data, a maximum of the ionic current data, and a standard deviation of the ionic current data, in any combination and order.

In some embodiments, the action of detecting the capture event in response, at least in part, to determining that the features of the set of features are within the expected range for each feature, further includes: determining an amount of time for which an output strand associated with the capture event remained within the nanopore; and detecting the capture event in response, at least in part, to determining that the amount of time is greater than a threshold amount of time.

In some embodiments, each output strand of the plurality of output strands includes a barcode sequence selected from a set of barcode sequences. Determining concentrations of each of the plurality of output strands is based on quantifying the plurality of capture events associated with each barcode sequence over time. In some embodiments, the rate of capture is determined by determining the time between capture events of the barcode. In some embodiments, the rate of capture is determined by determining the number of capture events associate with each barcode in a defined period of time. The barcode sequence is determined for the output strand associated with the capture events and the corresponding rate of capture over time is associated with the sequence of the output strand.

In some embodiments, determining the barcode sequence of the output strand associated with the capture event includes: providing the ionic current signal data associated with the capture event as input to a machine learning model trained to identify the barcode sequences from the ionic current signal data. In some embodiments, the machine learning model is a non-neural network model. Exemplary, non-limiting non-neural network models include Vector Machine, Random Forest, and the like. In some embodiments, the machine learning model is a neural network model. An exemplary neural network model is a convolutional neural network (CNN).

In some embodiments, the barcode sequences of the set of barcode sequences can be unique random sequences. In other embodiments, the barcode sequences of the set of barcode sequences are designed to be distinguishable from each other by the machine learning model.

### General Definitions

Unless specifically defined herein, all terms used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E., et al. (eds.), Modern Proteomics—Sample Preparation, Analysis and Practical Applications in Advances in Experimental Medicine and Biology, Springer International Publishing, 2016, and Comai, L, et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or” in the claims is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense, which is to indicate, in the sense of “including, but not limited to.” Words using the singular or plural number also include the plural and singular number, respectively. The word “about” indicates a number within range of minor variation above or below the stated reference number. For example, in some embodiments “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are provided for the purpose of illustrating, not limiting, the disclosure.

### Example 1

This example describes the development of an alternative detection mechanism for DNA strand displacement (DSD) circuits to realize the scalable potential of such technologies.

Introduction

A scalable method was developed for signal detection by adapting nanopore sensing technology to dynamically detect DSD circuit kinetics, enabling fast and scalable circuit readout on an inexpensive, commercially available device.

For proof of concept, the Oxford Nanopore MinION® nanopore device was used as an exemplary nanopore system to detect and monitor DSD circuit kinetics. The MinION® is a small, portable device designed to sequence DNA and RNA by electrophoretically driving nucleic acid strands through an array of CsgG protein pores and sensing the characteristic current blockade of each nucleotide. Translocation of strands through the pores is mediated by motor proteins (helicases). Ligation of these motor protein adapters to target strands must occur prior to sequencing, which makes this pipeline inconvenient for DSD circuit readout, especially when analysis of circuit kinetics is desired. Furthermore, strands in DSD circuits are usually too short (around 20-50 bp) to be reliably detected via conventional sequencing.

In the context of DNA computing, nanopore detection of a DNA circuit has been demonstrated using a micro-droplet system, wherein the target strand is electrophoretically pulled through a protein pore connecting two droplets. However, studies have not yet shown these systems to be quantifiable, nor has their multiplexing potential been explored. Apart from DNA computing, nanopore technology has also been adapted for miRNA detection, facilitating disease diagnostics. Further studies have demonstrated that peptide nucleic acid (PNA) and polyethylene glycol (PEG) probes are effective at targeting specific miRNAs for nanopore detection. However, it remains challenging to detect multiple miRNAs using these probes due to the high similarity of their nanopore signals, which also precludes quantification.

To address these challenges, a new approach was developed for capturing and sensing DSD circuit strands using nanopore sensing technology, without any modification to the off-the-shelf Oxford Nanopore MinION® device. DNA output strands were coupled with streptavidin tags, blocking the strands from fully translocating the nanopore, allowing a static read of the output strand residing within the pore. This method was first demonstrated on a seesaw circuit. The reaction measured from nanopore showed similar kinetics to that measured from a traditional plate reader. Subsequently, ten orthogonal DNA barcodes were designed to be simultaneously sensed by a MinION® and distinguished by a Convolutional Neural Network (CNN) with ˜80% accuracy. Finally, the method was demonstrated by reading ten orthogonal seesaw gates.

Results

A system was designed in which an output strand of a DSD circuit is blocked from fully translocating the pore once it is captured, enabling a static read of the strand segment residing within the pore. This is accomplished by biotinylating the 3′ end of the output strand and running the sample with streptavidin. Single-stranded output strands are electrophoretically captured in the pores but are sterically hindered from fully translocating to the other side by the bound streptavidin (FIG. 1A). After 10 seconds of a “forward” voltage polarity, the voltage polarity is reversed for 5 seconds (using a custom MinION® script), ejecting the captured strand and freeing the pore to sample a new strand from solution. To confirm that output strand captures generate a distinguishable change (e.g., drop) in the detectable ionic current, a sample of 1 uM biotinylated single-stranded output with 4 uM streptavidin was run on the MinION® (R9.4.1 flow cell). Streptavidin was purposefully incorporated at four times the concentration of output strand to minimize the probability of two output strands binding to the same streptavidin protein. The resulting plot of raw nanopore current over time clearly shows locations where the current drops below open pore value within a 15-second voltage flip window (FIG. 1B). These drops, or “blockades,” correspond to capture events where the barcode can be is “read” from the raw current signal.

Capture events can be the result of any molecule entering or blocking the pore, whether it is streptavidin-bound output strand, streptavidin itself, or background noise. Thus, the next step was to isolate capture events attributed to the streptavidin-bound output strand. First, a data analysis pipeline was built that processes the nanopore raw signal, locates capture events, and calculates five features (mean, median, minimum, maximum, standard deviation) of the ionic current during each capture event. A distribution plot of mean fractional current shows that captured output strands occupy a unique signal space from streptavidin captures (FIG. 1C). A fractional current is defined as the blockaded current during a capture event (I_{b}) divided by the open pore current (Iₒ). Distinct peaks in the output strand distribution were observed for the other four signal features as well (not shown). A filter was then designed to isolate putative output strand capture events by checking whether their five signal features are within the expected range. A length filter was also additionally applied to discard captures that remained in the pore for less than two seconds. This removes noise captures (resulting from small molecules passing through the pore) and ensures there is enough data from each capture event for downstream analysis.

Next, the output strand that would enable analysis of circuit kinetics was quantified. It was reasoned that output strand concentration would be correlated to the average time between capture events, with a longer time between captures corresponding to a lower concentration. To calculate time between captures, the end time of a given capture was subtracted from the start time of the subsequent capture. Additionally, any time periods were subtracted where the pore was occupied with a non-output strand capture to minimize the effect of background noise on the predicted concentration. The average of these times between captures was calculated from an aggregate list of total time between captures from all functional pores in a given experiment. To convert from average time between captures to concentration, a standard curve was used that was generated from a series of titration experiments with known output strand concentrations (FIG. 1D). The curve represents an average of three titration experiments with unique circuit outputs.

With the ability to quantify strand concentrations, the next goal was to measure the kinetics of a full DSD circuit on the nanopore. The analyte for this experiment included a seesaw gate, streptavidin, and fuel which regenerates the input strand after the input binds to the gate (FIG. 1A). Immediately prior to loading on the MinION®, input strand was added to initiate the reaction. The analyte was then introduced to the MinION® and ionic current data was collected over the course of four hours. The average time between captures was calculated for each five-minute interval throughout the run and converted into a predicted concentration via the standard curve. The result was a concentration vs. time kinetics curve for the DSD circuit (FIG. 1E). To provide a comparison, the same circuit was quantified using a fluorescence reporter on a plate reader spectrometer. As shown in FIG. 1E, the curve showing kinetics with input strands in a given circuit as quantified on the nanopore was comparable to the corresponding kinetics curve on a fluorescence plate reader, indicating that the nanopore can accurately characterize circuit behavior. Having proven it is possible to quantify a single DSD circuit using nanopore sensing, the next step was to assess the potential to use the nanopore-based reporting in multiplexed circuits. It was hypothesized that output strands with unique barcode sequences could be distinguished from each other by characteristic features in their capture event signals, similar to how isolated streptavidin-bound output strand captures were distinguished from noise captures. Prior to designing more circuits however, the precise region on the output strand that is sensed by the nanopore was mapped. This was accomplished by creating single-nucleotide mutations in the output strand sequence within a nine-nucleotide window directly upstream of the biotinylated 3′ end. The fractional current features of each of these nine mutants were compared to that of the original output strand, demonstrating that only mutations within a six-nucleotide subset of that window generated a significant change in fractional current relative to the original (FIG. 1F). This six-nucleotide region was designated as the strand's barcode.

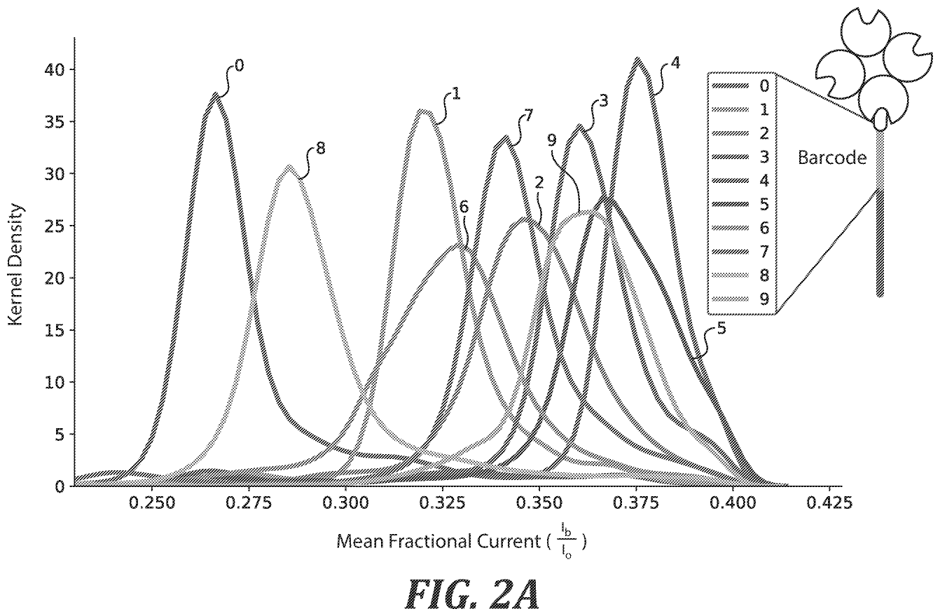

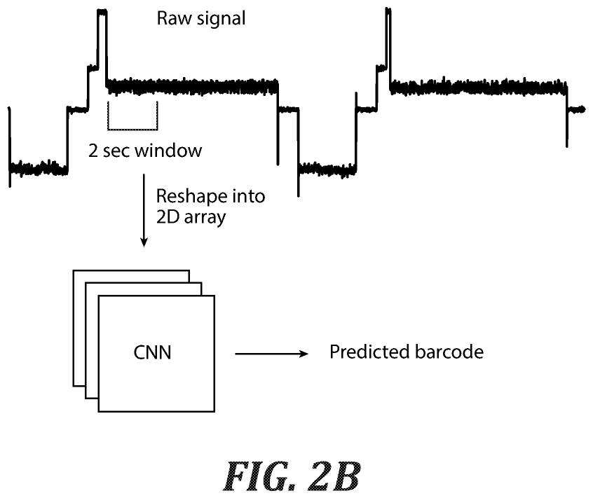

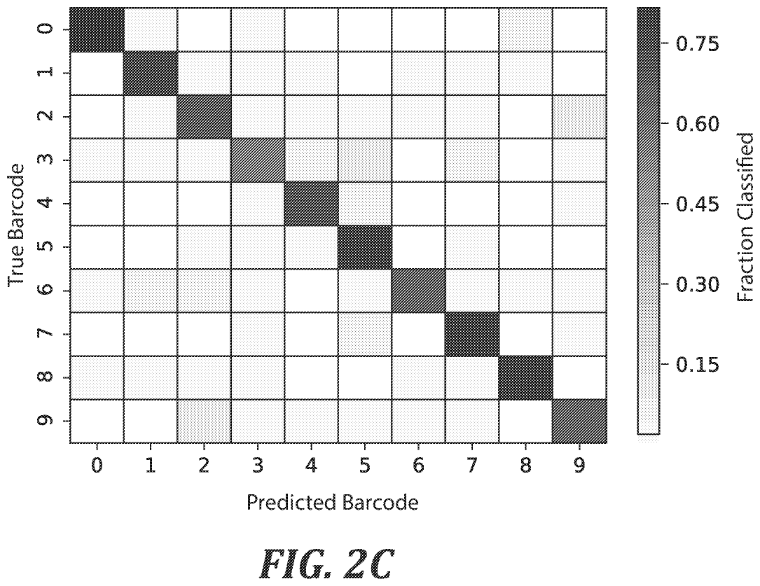

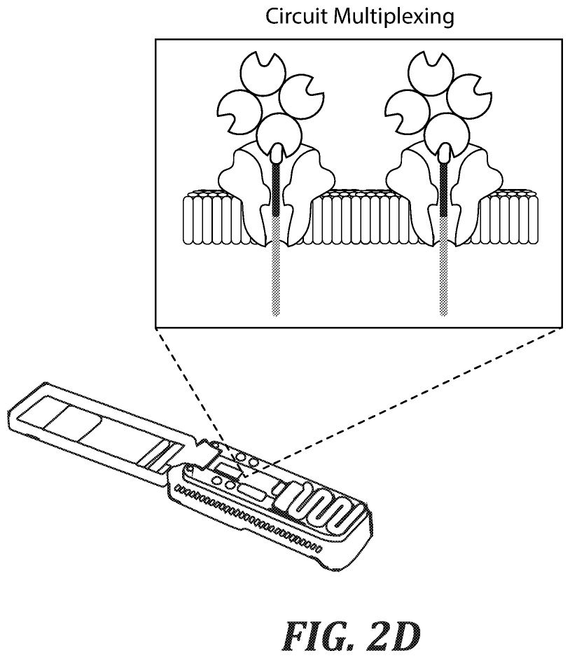

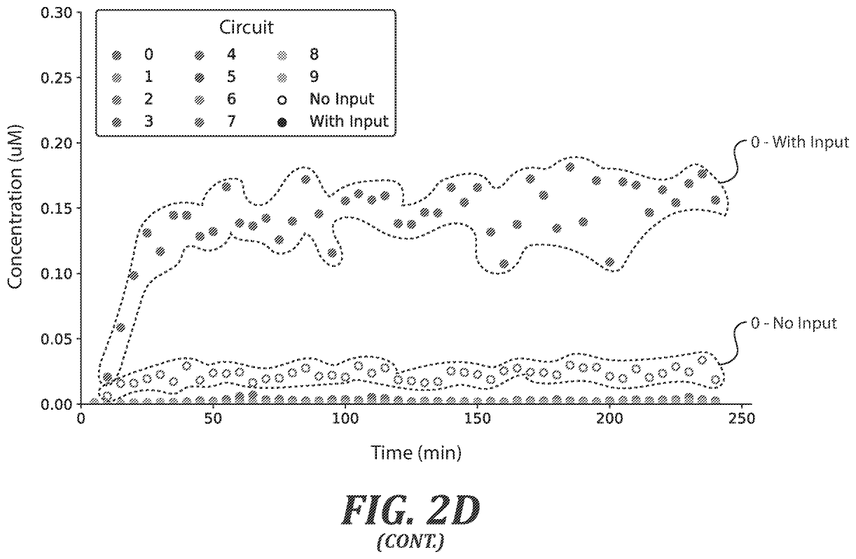

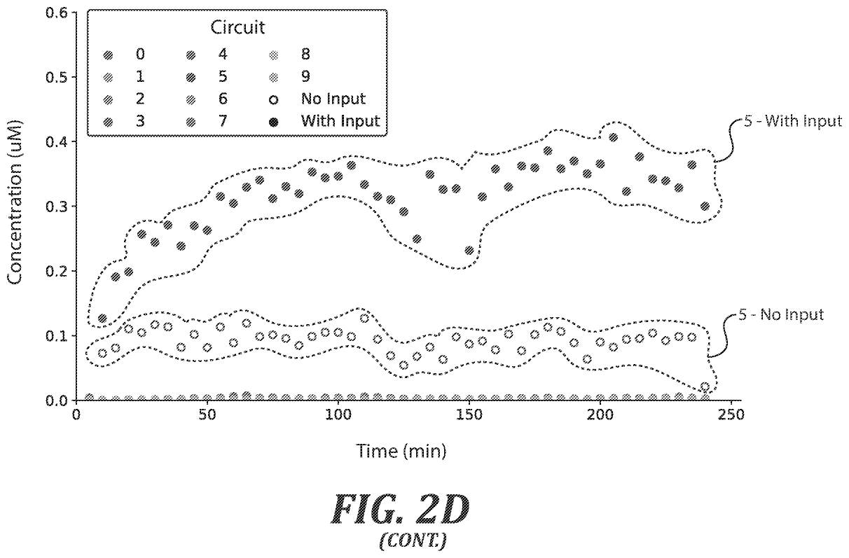

Ten unique circuits were designed, each with a semi- or pseudo-randomly chosen barcode on the output strand. A combined distribution plot of the fractional mean nanopore current for each of these barcodes shows that while some barcodes have distinct peaks indicating high separability, others form a cluster that could make them difficult to distinguish based on mean current alone (FIG. 2A). Thus, to better discriminate between the signals generated by different barcodes, a Convolutional Neural Network (CNN) machine learning classifier was built to conduct feature extraction on a two-second window of each capture event (FIG. 2B). The classifier was trained using data collected from separate nanopore experiments for each barcode. Applying the classifier model to a test set yielded a prediction accuracy of up to 72% (FIG. 2C). This model was subsequently used to quantify the kinetics of a two-circuit multiplexed reaction (FIG. 2D). The classifier was successfully able to distinguish the barcodes that were present from the other barcodes in the random barcode set.

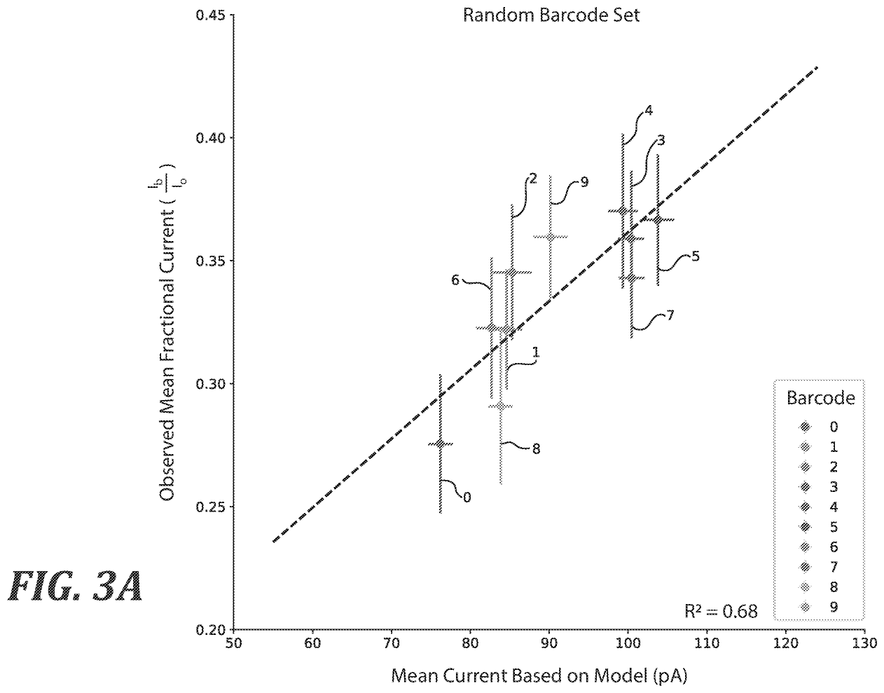

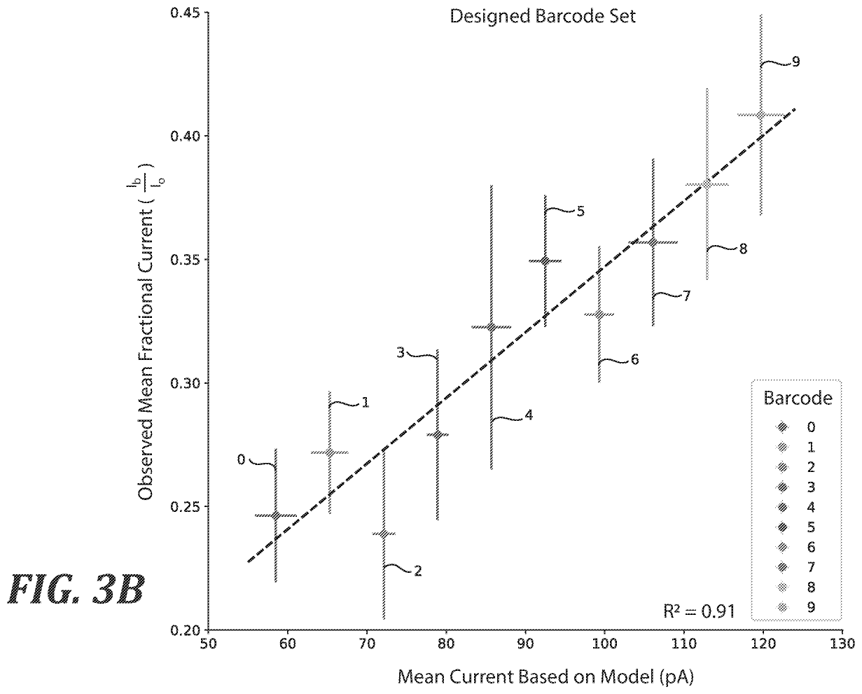

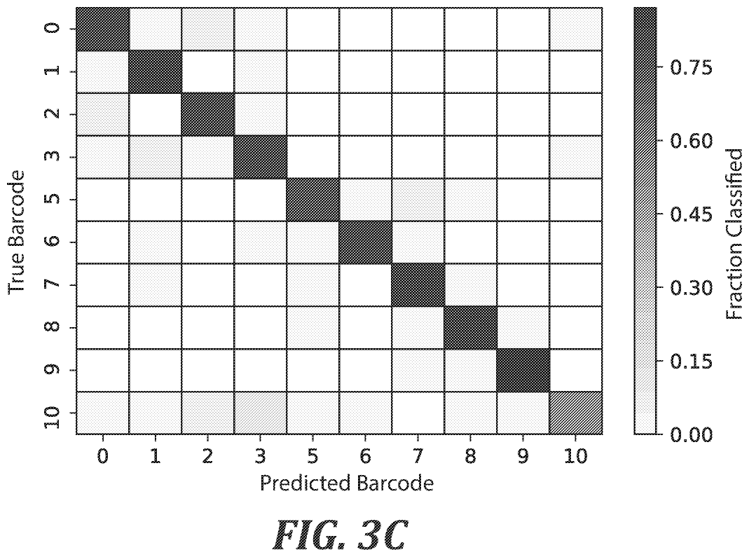

To facilitate performance of the classifier to distinguish between barcodes, a new set of barcodes were introduced that were designed to be highly distinguishable in the particular nanopore platform (i.e., MinION® from Oxford Nanopore Technology (ONT)), as opposed to using randomly generated barcodes. The extent of nanopore signal space that a six-nucleotide barcode could feasibly occupy was investigated. To do this, a publicly available online table of predicted nanopore signals for all possibilities of 6mers from ONT's code repository was consulted (://github.com/nanoporetech/kmer models). First, it was established that the ONT model's predicted signals could serve as an appropriate approximation of the signals observed in the present experiments. The observed mean fractional current for the ten random barcodes were plotted against the corresponding mean signals predicted by the ONT model, which resulted in a R\hat{ }2 of 0.68 (FIG. 3A). This R\hat{ }2 value is significant, but the observed signal has a noticeably higher variation than the model's signal. This could be due to the fact that static captures were used for the observed signals as opposed translocated captures that were used to generate ONT's predicted signals. After affirming the model's correlation with the experimental observations, ten new circuit barcodes were designed by choosing the ten 6mers with the most separable signal means from the model for this nanopore platform. Similar to the random barcode set, the observed mean fractional current for this designed barcode set were plotted against the corresponding mean signals predicted by the model (FIG. 3B). Compared to the random set, the designed set barcodes occupy a wider range of signal space and are generally individually separable (i.e., there are no clusters). Interestingly, the R\hat{ }2 value was much higher at 0.91, although the observed signals still had very high variation. These data were used to train the CNN model, which was able to achieve an accuracy of ˜80% when distinguishing among the barcodes (FIG. 3C).

Methods and Materials

MinION® Experiments

All MinION® runs were performed using R9.4.1 flow cells (Oxford Nanopore Technologies). All runs were configured at a temperature of 30° C., bias voltage of −180 mV, sampling frequency of 10 kHz, and static flip frequency of 15 sec. Along with the appropriate amount of circuit components and streptavidin (depending on the experiment), all analytes contained 50 uL of 4×C17 buffer (2 M KCl, 100 mM HEPES, pH 8) and enough water to fill to a total reaction volume of 200 uL. Analytes were pipetted into the flow cell priming port. If running multiple analytes on the same flow cell, the flow cell was washed with 3 mL 1×C17 for 5 min between analytes. When not in use, flow cells were stored at 4° C. in C18 buffer (150 mM potassium ferrocyanide, 150 mM potassium ferricyanide, 25 mM potassium phosphate, pH 8).

Circuit Design and Construction

All DSD circuit components were synthesized at Integrated DNA Technologies. All components were stored at −20° C. in the long-term and 4° C. in the short term (no more than two weeks). Sequence for the DSD circuit component strands are set forth in Table 1, and the semi-random and rationally designed barcode sequences that were integrated into the output strands are set forth in Table 2.

TABLE 1
Sequences for elements of the nucleic acid circuits,
including seesaw gate (partner strand), output strand, input strand,
and fuel strand for all ten circuits used for the semi-
random and designed barcode sets. The barcode region is
represented by the underlined N domain.

| Circuit | | | SEQ ID |
|---|---|---|---|
| No. | Strand | Sequence | NO. |
| 0 | Gate | AGAGTGTGGAGTTGATAGGAGAG | 1 |
| | Output | ACACCTTACTCTCTACTCTCCTATCAACTCCACAT | 2 |
| | | TTTTTNNNNNNTT/3BioTEG/ | |
| | Input | CCTATCAACTCCACACTCTCACTAATTCTACATC | 3 |
| | Fuel | TACCTCATTCAAACTCTCTCCTATCAACTCCACA | 4 |
| 1 | Gate | AGAGAATAATGGTTGTAGGAGAG | 5 |
| | Output | CTTCTTATACCACACCTCTCCTACAACCATTATTT | 6 |
| | | TTTTTNNNNNNCA/3BioTEG/ | |
| | Input | CCTACAACCATTATTCTCTCAATCTACCAAACTC | 7 |
| | Fuel | ACAAATACCTCATCCCTCTCCTACAACCATTATT | 8 |
| 2 | Gate | AGAGTAAGTATAGAGGTGAAGAG | 9 |
| | Output | CAACTACAATCTCTCCTCTTCACCTCTATACTTAT | 10 |
| | | TTTTTNNNNNNTA/3BioTEG/ | |
| | Input | TCACCTCTATACTTACTCTTCCTTCATCTTCTAC | 11 |
| | Fuel | TCTCCAATTTCAACTCTCTTCACCTCTATACTTA | 12 |
| 3 | Gate | AGAGAGGATTAGGATAGTGAGAG | 13 |
| | Output | AACCACATTAACCTTCTCTCACTATCCTAATCCTT | 14 |
| | | TTTTTNNNNNNCC/3BioTEG/ | |
| | Input | CACTATCCTAATCCTCTCTAAACCTTACCACCAC | 15 |
| | Fuel | CCTTCTCAACTCCTCCTCTCACTATCCTAATCCT | 16 |
| 4 | Gate | AGAGGGTGTTTAGAGTTTAAGAG | 17 |
| | Output | TTATCCAACTCACTACTCTTAAACTCTAAACACC | 18 |
| | | TTTTTTNNNNNNAT/3BioTEG/ | |
| | Input | TAAACTCTAAACACCCTCTAATAACACCTCCTAA | 19 |
| | Fuel | CTCTTCTTTCCAAACCTCTTAAACTCTAAACACC | 20 |
| 5 | Gate | AGAGAAAGTGATAAGATGGAGAG | 21 |
| | Output | TCTTTCACCTCACATCTCTCCATCTTATCACTTTT | 22 |
| | | TTTTTNNNNNNCA/3BioTEG/ | |
| | Input | CCATCTTATCACTTTCTCTATTACTTCCTACACC | 23 |
| | Fuel | ATCCTCCTTCCATCCCTCTCCATCTTATCACTTT | 24 |
| 6 | Gate | AGAGGGTATTAGTTAGGTAAGAG | 25 |
| | Output | TCCATTTCATTTCACCTCTTACCTAACTAATACCT | 26 |
| | | TTTTTNNNNNNAA/3BioTEG/ | |
| | Input | TACCTAACTAATACCCTCTCTCCATAACATTCCA | 27 |
| | Fuel | ACTTCTAACAACTACCTCTTACCTAACTAATACC | 28 |
| 7 | Gate | AGAGTGTTAGTAGTAGAGTAGAG | 29 |
| | Output | AAATTCTATCCACTCCTCTACTCTACTACTAACA | 30 |
| | | TTTTTTNNNNNNCC/3BioTEG/ | |
| | Input | ACTCTACTACTAACACTCTTCTACATCCACATCT | 31 |
| | Fuel | CACTCAATAACTACCCTCTACTCTACTACTAACA | 32 |
| 8 | Gate | AGAGGTATAAAGGAGTTTGAGAG | 33 |
| | Output | TAACTCTACCACAAACTCTCAAACTCCTTTATAC | 34 |
| | | TTTTTTNNNNNNTA/3BioTEG/ | |
| | Input | CAAACTCCTTTATACCTCTCTCTACTCATCTTCC | 35 |
| | Fuel | CCACCTCCATCTATACTCTCAAACTCCTTTATAC | 36 |
| 9 | Gate | AGAGTGGTAAGGTAGTTAAAGAG | 37 |
| | Output | CTAACAAACTTTACCCTCTTTAACTACCTTACCA | 38 |
| | | TTTTTTNNNNNNTC/3BioTEG/ | |
| | Input | TTAACTACCTTACCACTCTACATTCCTTCTAATC | 39 |
| | Fuel | ATTTACATCTCAACCCTCTTTAACTACCTTACCA | 40 |

TABLE 2
Output strand barcodes from the
semi-random set and designed set that were
incorporated in the output strands at the
domains represented by the underlined N as
set forth in Table 1.

| Circuit | Semi-Random | |
|---|---|---|
| No. | Set Barcode | Designed Set Barcode |
| 0 | CAAATA | GGGTTC |
| 1 | TCATAC | TGATTG |
| 2 | ATATCT | AGAGTT |
| 3 | CTCCAC | AGAGGA |
| 4 | ATCTAA | ATATCA |
| 5 | CTCAAA | TTCTGT |
| 6 | AAATAC | AGCCTC |
| 7 | TCCAAC | GATACT |
| 8 | CAAAAC | TCTCTG |
| 9 | ACCTCC | AATCAA |

Circuit amplifiers were constructed by mixing 4 nmoles of the output strand with 4 nmoles of the toehold strand in 0.8×C17. Strands were annealed in a thermocycler starting at 95° C. and decreasing 1° C. every 1 min cycle for 75 cycles. The annealed product was then gel purified using 10% ND-PAGE. Purified product was eluted from the gel using 1×C17.

Fluorescence reporters were constructed by mixing 1.3× of the quencher strand with 1× of the fluorophore strand in 1× C17. Strands were annealed using the same thermocycler protocol for circuit amplifier construction. No purification was performed on the annealed product.

Titration Experiments

The standard curve is an average of average time between captures for three different output strand titration experiments. In each experiment, the output strand was run on the MinION® at concentrations of 0.02, 0.1, 0.2, 0.5, 1 uM. For all concentrations, 4 uM of streptavidin was added to the analyte. To ensure enough captures were collected for data analysis at low concentrations, the 0.02 uM analyte was run for 20 min, the 0.1 uM analyte for 15 min, and the rest of the analytes for 10 min. 5 min washes were conducted between each analyte.

Data Analysis Pipeline

The data analysis pipeline begins by isolating capture events from raw nanopore data. A capture event occurs when the nanopore current drops to 70% or below of its open pore level for longer than one millisecond.

Summary

Previous work demonstrated that single-stranded DNA oligos can be electrophoretically driven through nanopore sensors by an applied voltage. However, translocation is typically too fast to resolve meaningful amounts of sequence information. In the reporter strategy developed here, detection of select DNA circuit elements is accomplished by attaching anchor constructs to the potential output strands. The anchor construct can slow or completely arrest translocation of the output strand in the nanopore allowing recordation of sufficient current signal to be able to identify and differentiate the barcode sequence that influences the change in current through the nanopore. To demonstrate the proof of concept, the 5′ end of desired strands were biotinylated and streptavidin added to the reaction. Streptavidin complexes with the biotinylated strands and, because of its high affinity for biotin and large tertiary structure, streptavidin-bound ssDNA that are electrophoretically captured in the nanopore are prevented from completely translocating through the pore (See, e.g., FIGS. 1A and 1F). This produces a sequence-dependent current blockage and makes it possible to obtain a static read of the portion of the strand that is residing within the pore (the barcode) (see, e.g., FIG. 1B). The voltage polarity of the pore can then be reversed (or “flipped), ejecting the captured strand, and freeing the pore to sample a new strand from solution. Different strands can be uniquely barcoded by changing the sequence of the strand in the capture region, which renders each output strand with different barcodes detectable and identifiable (see, e.g., FIG. 2A). Quantification is achieved by relating the average time between capture events to concentration using a standard curve (FIG. 1D). This calculation can be repeated across regular time intervals to generate a kinetics curve for a given reaction (FIG. 1E). When multiplexing, an unknown barcode capture is identified from its raw current data using a machine learning classifier that was trained on data from known barcode strand sequence runs. The algorithm uses a combination of signal features (e.g. mean, median, minimum, maximum, standard deviation) to make a classification. In this way a multiplex of DNA displacement circuits can be interrogated in parallel to detect and quantify a multitude is DNA components in a DNA computing method.

While illustrative embodiments have been illustrated and described, it will be appreciated that various changes can be made therein without departing from the spirit and scope of the invention.

1. A method of detecting a nucleic acid strand displacement circuit in a nanopore system comprising a nanopore disposed between a first conductive liquid medium and a second conductive liquid medium, wherein the nanopore comprises a tunnel that provides liquid communication between the first conductive liquid medium and the second conductive liquid medium, the method comprising:
contacting a double stranded complex with an input strand, wherein the double stranded complex comprises a partner strand hybridized along a first portion of the partner strand to an output strand along a portion of the output strand,
permitting hybridization of the input strand to the partner strand along a second portion of the partner strand that partially overlaps with the first portion of the partner strand, thereby displacing the output strand from the double stranded complex;
translocating the displaced output strand through the nanopore from the first conductive liquid medium towards the second conductive liquid medium;
measuring an ion current through the nanopore when the displaced output strand is in the tunnel to provide a current pattern corresponding to a portion of the displaced output strand; and
associating the current pattern with the input strand that displaced the output strand, thereby detecting the nucleic acid strand displacement circuit.

2. The method of claim 1, wherein the partner strand comprises a toehold sequence and a partner sequence, and the output strand comprises a first domain with a sequence that hybridizes to the partner sequence and a second domain with a barcode sequence

3. The method of claim 2, wherein the barcode sequence does not hybridize with any portion of the partner strand.

4. The method of claim 2, wherein the input strand comprises a sequence that hybridizes to the toehold sequence and at least a portion of the partner sequence of the partner strand.

5. The method of claim 1, wherein the output strand comprises a barcode sequence and an anchor moiety, wherein the anchor moiety has dimensions that exceed the diameter of the tunnel preventing passage through the nanopore, and wherein the anchor moiety is configured to arrest or slow translocation when the anchor moiety contacts an outer surface or inner constriction region of the nanopore resulting in the barcode sequence of the displaced output strand being disposed within the tunnel of the nanopore for a time sufficient to measure the ion current.

6. The method of claim 5, further comprising adding the anchor moiety prior to the translocation step.

7-8. (canceled)

9. The method of claim 5, wherein the anchor moiety is a protein with tertiary structure comprising dimensions that exceed the diameter of the tunnel and does not pass through the nanopore or the anchor moiety is or comprises a nucleic acid moiety with secondary structure.

10-11. (canceled)

12. The method of claim 1, wherein the contacting and permitting hybridization steps are performed in the first conductive liquid medium of the nanopore system.

13. The method of claim 1, wherein the translocating step comprises applying an electrical potential between the first conductive liquid medium and the second conductive liquid medium to promote translocation of the displaced output strand through the nanopore towards the second conductive liquid medium.

14. The method of claim 13, further comprising reversing polarity of the electrical potential after measuring the ion current in a manner sufficient to reverse translocation direction towards the first conductive liquid medium, thereby causing the displaced output strand to exit the nanopore into the first conductive liquid medium.

15. The method of claim 14, wherein the method comprises contacting a plurality of the double stranded complex with a plurality of the input strand in a common reaction volume, wherein the method further comprising repeating the steps of translocation, measuring ion current, and reversing polarity one or more times, and wherein the method further comprises quantifying over time the capture events resulting in current patterns corresponding to the displaced output strand.

16. The method of claim 15, further comprising correlating time and/or a rate between capture events with the concentration of the associated input strand in the common reaction volume.

17. (canceled)

18. The method of claim 1, comprising performing the method for a plurality of distinct nucleic acid strand displacement circuits in a common reaction volume, wherein the common reaction volume comprises a plurality of distinct double stranded complexes with distinct sequences in their respective second portion of the partner strand.

19. (canceled)

20. The method of claim 1, wherein associating the current pattern from the output strand with the input strand to detect the one or more nucleic acid strand displacement circuits comprises:
(a) providing the current pattern, or one or more signal parameters extracted from the current pattern, to a machine learning model to determine a unique digital fingerprint, and
(b) determining the association of the unique digital fingerprint with the input strand that displaced the output strand.

21. The method of claim 20, wherein the one or more signal parameters of the current pattern include mean current, median current, minimum current, maximum current, and/or standard deviation of current, in any combination.

22-25. (canceled)

26. The method of claim 1, wherein the nanopore, or
portion thereof in contact with the first conductive liquid medium, has a net neutral or net positive charge.

27-28. (canceled)

29. The method of claim 1, wherein the nanopore system comprises one or more electrodes in contact with the first conductive liquid medium and the second conductive liquid medium configured to generate an electrical potential across the barrier.

30. The method of claim 1, wherein the method is a molecular diagnostic method wherein detection of a nucleic acid strand displacement circuit is indicative of the presence or amount of a target biomarker.

31. A system, comprising:
a nanopore system comprising:
a nanopore disposed in a barrier defining a cis side and a trans side, wherein the cis side comprises a first conductive liquid medium and the trans side comprises a second conductive liquid medium, and wherein the nanopore comprises a tunnel that provides liquid communication between the cis side and the trans side;
a controllable voltage source connected to the cis side and the trans side by one or more electrodes, wherein the controllable voltage source is configured to generate an electrical potential across the barrier; and
a data acquisition device operable to detect an ion current through the nanopore;
a plurality of distinct double stranded nucleic acid complexes, wherein each double stranded nucleic acid complex comprises a partner strand hybridized along a first portion of the partner strand to an output strand along a portion of the output strand, wherein the nanopore system is operative to individually translocate the output strands in single stranded format from the first conductive liquid medium toward the second conductive liquid medium through the tunnel and detect an ion current through the nanopore while each output strand is in the tunnel; and
a computing system communicatively coupled to the nanopore system, the computing system including logic that, in response to execution by at least one processor of the computing system, causes the computing system to perform actions for analyzing a current pattern from the detected ion current, the actions comprising:
receiving from the nanopore system a plurality of signals, wherein the plurality of signals represent an ion current pattern detected in the nanopore while each output strand is in the tunnel; and
providing the plurality of signals, or the one or more signal parameters extracted therefrom, to a machine learning model to determine a unique digital fingerprint corresponding to a unique sequence of the output strand.

32-45. (canceled)

46. A non-transitory computer-readable medium having computer-executable instructions stored thereon that, in response to execution by one or more processors of a computing system, cause the computing system to perform actions for detecting a result of a nucleic acid-based computation, the actions comprising:
receiving, by the computing system from a nanopore system, ionic current signal data generated while processing a plurality of output strands through at least one nanopore;
detecting, by the computing system, a plurality of capture events within the ionic current signal data;
determining, by the computing system, concentrations of the plurality of output strands based on the plurality of capture events over time; and
providing, by the computing system, the concentrations of the plurality of output strands as an output of the DNA-based computation.

47-54. (canceled)


SEQUENCE LISTING (40 sequences)

SEQ ID NO: 1 - DNA, 23 bp, Artificial sequence
  Synthetic
agagtgtggagttgataggagag

SEQ ID NO: 2 - DNA, 48 bp, Artificial sequence
  Synthetic
  misc_feature (41)..(46): N is any nucleotide or abasic site
acaccttactctctactctcctatcaactccacattttttnnnnnntt

SEQ ID NO: 3 - DNA, 34 bp, Artificial sequence
  Synthetic
cctatcaactccacactctcactaattctacatc

SEQ ID NO: 4 - DNA, 34 bp, Artificial sequence
  Synthetic
tacctcattcaaactctctcctatcaactccaca

SEQ ID NO: 5 - DNA, 23 bp, Artificial sequence
  Synthetic
agagaataatggttgtaggagag

SEQ ID NO: 6 - DNA, 48 bp, Artificial sequence
  Synthetic
  misc_feature (41)..(46): N is any nucleotide or abasic site
cttcttataccacacctctcctacaaccattattttttttnnnnnnca

SEQ ID NO: 7 - DNA, 34 bp, Artificial sequence
  Synthetic
cctacaaccattattctctcaatctaccaaactc

SEQ ID NO: 8 - DNA, 34 bp, Artificial sequence
  Synthetic
acaaatacctcatccctctcctacaaccattatt

SEQ ID NO: 9 - DNA, 23 bp, Artificial sequence
  Synthetic
agagtaagtatagaggtgaagag

SEQ ID NO: 10 - DNA, 48 bp, Artificial sequence
  Synthetic
  misc_feature (41)..(46): N is any nucleotide or abasic site
caactacaatctctcctcttcacctctatacttattttttnnnnnnta

SEQ ID NO: 11 - DNA, 34 bp, Artificial sequence
  Synthetic
tcacctctatacttactcttccttcatcttctac

SEQ ID NO: 12 - DNA, 34 bp, Artificial sequence
  Synthetic
tctccaatttcaactctcttcacctctatactta

SEQ ID NO: 13 - DNA, 23 bp, Artificial sequence
  Synthetic
agagaggattaggatagtgagag

SEQ ID NO: 14 - DNA, 48 bp, Artificial sequence
  Synthetic
  misc_feature (41)..(46): N is any nucleotide or abasic site
aaccacattaaccttctctcactatcctaatcctttttttnnnnnncc

SEQ ID NO: 15 - DNA, 34 bp, Artificial sequence
  Synthetic
cactatcctaatcctctctaaaccttaccaccac

SEQ ID NO: 16 - DNA, 34 bp, Artificial sequence
  Synthetic
ccttctcaactcctcctctcactatcctaatcct

SEQ ID NO: 17 - DNA, 23 bp, Artificial sequence
  Synthetic
agagggtgtttagagtttaagag

SEQ ID NO: 18 - DNA, 48 bp, Artificial sequence
  Synthetic
  misc_feature (41)..(46): N is any nucleotide or abasic site
ttatccaactcactactcttaaactctaaacaccttttttnnnnnnat

SEQ ID NO: 19 - DNA, 34 bp, Artificial sequence
  Synthetic
taaactctaaacaccctctaataacacctcctaa

SEQ ID NO: 20 - DNA, 34 bp, Artificial sequence
  Synthetic
ctcttctttccaaacctcttaaactctaaacacc

SEQ ID NO: 21 - DNA, 23 bp, Artificial sequence
  Synthetic
agagaaagtgataagatggagag

SEQ ID NO: 22 - DNA, 48 bp, Artificial sequence
  Synthetic
  misc_feature (41)..(46): N is any nucleotide or abasic site
tctttcacctcacatctctccatcttatcactttttttttnnnnnnca

SEQ ID NO: 23 - DNA, 34 bp, Artificial sequence
  Synthetic
ccatcttatcactttctctattacttcctacacc

SEQ ID NO: 24 - DNA, 34 bp, Artificial sequence
  Synthetic
atcctccttccatccctctccatcttatcacttt

SEQ ID NO: 25 - DNA, 23 bp, Artificial sequence
  Synthetic
agagggtattagttaggtaagag

SEQ ID NO: 26 - DNA, 48 bp, Artificial sequence
  Synthetic
  misc_feature (41)..(46): N is any nucleotide or abasic site
tccatttcatttcacctcttacctaactaataccttttttnnnnnnaa

SEQ ID NO: 27 - DNA, 34 bp, Artificial sequence
  Synthetic
tacctaactaataccctctctccataacattcca

SEQ ID NO: 28 - DNA, 34 bp, Artificial sequence
  Synthetic
acttctaacaactacctcttacctaactaatacc

SEQ ID NO: 29 - DNA, 23 bp, Artificial sequence
  Synthetic
agagtgttagtagtagagtagag

SEQ ID NO: 30 - DNA, 48 bp, Artificial sequence
  Synthetic
  misc_feature (41)..(46): N is any nucleotide or abasic site
aaattctatccactcctctactctactactaacattttttnnnnnncc

SEQ ID NO: 31 - DNA, 34 bp, Artificial sequence
  Synthetic
actctactactaacactcttctacatccacatct

SEQ ID NO: 32 - DNA, 34 bp, Artificial sequence
  Synthetic
cactcaataactaccctctactctactactaaca

SEQ ID NO: 33 - DNA, 23 bp, Artificial sequence
  Synthetic
agaggtataaaggagtttgagag

SEQ ID NO: 34 - DNA, 48 bp, Artificial sequence
  Synthetic
  misc_feature (41)..(46): N is any nucleotide or abasic site
taactctaccacaaactctcaaactcctttatacttttttnnnnnnta

SEQ ID NO: 35 - DNA, 34 bp, Artificial sequence
  Synthetic
caaactcctttatacctctctctactcatcttcc

SEQ ID NO: 36 - DNA, 34 bp, Artificial sequence
  Synthetic
ccacctccatctatactctcaaactcctttatac

SEQ ID NO: 37 - DNA, 23 bp, Artificial sequence
  Synthetic
agagtggtaaggtagttaaagag

SEQ ID NO: 38 - DNA, 48 bp, Artificial sequence
  Synthetic
  misc_feature (41)..(46): N is any nucleotide or abasic site
ctaacaaactttaccctctttaactaccttaccattttttnnnnnntc

SEQ ID NO: 39 - DNA, 34 bp, Artificial sequence
  Synthetic
ttaactaccttaccactctacattccttctaatc

SEQ ID NO: 40 - DNA, 34 bp, Artificial sequence
  Synthetic
atttacatctcaaccctctttaactaccttacca